(19) United States
(12) Patent Application Publication
(10) Pub. No.: US 2026/0076579 A1
(43) Pub. Date: Mar. 19, 2026

(54) MEDICAL DEVICE MANAGEMENT UTILIZING ADAPTER-FACILITATED VIBRATION SENSORS

(71) Applicant: Stryker Corporation, Portage, MI (US)

(72) Inventors: Robert Greenewald, Snohomish, WA (US); Brendan Greetham, Redmond, WA (US)

(73) Assignee: Stryker Corporation, Portage, MI (US)

(21) Appl. No.: 19/332,711

(22) Filed: Sep. 18, 2025

Related U.S. Application Data
(60) Provisional application No. 63/696,241, filed on Sep. 18, 2024.

(51) Int. Cl. A61B 5/0205; A61B 5/00; A61B 5/021; A61B 5/08; A61B 5/113; A61B 5/318; A61B 7/04

(52) U.S. Cl. CPC A61B 5/0205; A61B 5/0002; A61B 5/113; A61B 5/318; A61B 5/681; A61B 5/746; A61B 7/04; A61B 5/021; A61B 5/0816

(57) ABSTRACT

Techniques for electronic devices including medical devices and non-medical devices are described. An example method includes receiving data from a wearable device via a connector in response to identifying that the wearable device is activated to detect a vibration. For instance, the vibration is associated with heartbeat data, breath data, NIBP data, and/or gastrointestinal data.

### CROSS-REFERENCE TO RELATED APPLICATION

This application claims priority to U.S. Provisional Application No. 63/696,241, which was filed on Sep. 18, 2024 and is incorporated by reference herein in its entirety.

### BACKGROUND

Various types of medical devices include capabilities to sense various types of characteristics associated with subjects, such as patients. Medical devices can be communicatively coupled with other devices, instruments, and peripherals. Some medical devices can include ports configured to communicatively couple to other devices, such as adapters, such that the medical devices can gain additionally types of functionality.

### BRIEF DESCRIPTION OF THE DRAWINGS

FIG. 1 illustrates an example environment in which vibration-sensing devices are configured to sense vibration and communicate signals via adapters that convert and route the signals to medical devices. The signals, for instance, include different types of sensor data generated based on the vibration.

FIGS. 2A-2D illustrates example vibration-sensing devices configured to gather and transmit, to adapters, signals with different types of sensor data, including heartbeat data, breath data, non-invasive blood pressure (NIBP) data, and gastrointestinal data.

FIG. 3 illustrates an example environment in which a vibration-sensing device senses vibration and communicates signals received by a monitor that identifies a level of ambient sound is above a threshold and generates an alarm.

FIG. 4 illustrates an example process for downloading, from devices having capabilities utilized to sense physiological parameters of subjects via vibration, data corresponding to the vibration.

FIG. 5 illustrates an example process for receiving data from wearable devices activated to detect vibration corresponding to breaths of subjects.

FIG. 6 illustrates an example of an external defibrillator configured to perform various functions described herein.

### DETAILED DESCRIPTION

Various implementations described herein relate to techniques for utilizing vibration-sensing devices exchanging data with medical devices. For instance, the vibration-sensing devices are configured to detect a vibration (e.g., a repetitive pattern of movement, such as acceleration) associated with a subject (e.g., a patient) and to communicate various types of health sensor data to the medical devices based on the vibration. Examples of the vibration-sensing devices include smart watches, smart phones, patch devices, wearable devices, implantable devices, and the like. In some cases, the sensor data is communicated via one or more adapters. In particular cases, the sensor data includes breath data, heartbeat data, NIBP data, gastrointestinal data, or the like. In some examples, the vibration is sensed by the vibration-sensing devices. Various signals exchanged between the vibration-sensing devices and the medical devices include wireless signals. In various cases, the adapters are configured to convert the communication protocols of the signals. In particular cases, the medical devices utilize the adapters to receive the signals and analyze the sensor data to identify information associated with the vibration. In particular cases, the information analyzed by the medical devices is associated with the breath data, heartbeat data, NIBP data, and/or gastrointestinal data.

In particular examples, a vibration-sensing device is incompatible with a communication protocol utilized by a medical device, such as a monitor-defibrillator. Thus, in these examples, the medical device is unable to directly receive and parse the sensor data from the wearable device.

In various implementations of the present disclosure, these and other problems are addressed by way of an adapter. In some examples, communication signals between the vibration-sensing device and the medical device are routed through an adapter. The adapter, for example, is configured to translate the communication signals between the communication protocols utilized by the vibration-sensing device and the medical device. Accordingly, the medical device can receive sensor data indicating vibrations detected by the vibration-sensing device. In some cases, the medical device utilizes the sensor data to identify information relevant to a health condition of the subject, or relevant to a treatment administered to the subject. For example, the medical device can utilize the sensor data to identify a breath of the subject, assisted ventilation administered to the subject, a heartbeat of the subject, a characteristic (e.g., a parameter of the characteristic) relevant to an NIBP measurement, a gastrointestinal state of the subject, or the like.

Implementations of the present disclosure are directed to improvements in the technical field of medical devices. Rather than requiring complex and/or cumbersome configurations, assemblies, and interconnections of multiple devices, implementations described herein enable utilization of wearable devices, and other vibration-sensing devices, with medical devices. By incorporating the corresponding capabilities into the wearable devices and adapters, the vibration-based sensor data can be efficiently, accurately, reliably, and safely communicated to, and analyzed by, the medical devices.

In contrast to wearable devices being unable to pair and exchange, with monitor-defibrillators, vibration-based sensor data via wireless signals, various techniques discussed herein enable the wearable devices to sense vibration and transmit the signals with the data based on the vibration in a convenient, quick, reliable, and simple way. For example, the medical devices utilize the adapter devices to transmit the signals with the data based on the vibration, which enables the monitor-defibrillators to analyze the vibration-based data in various settings, such as medical settings. In particular cases, the monitor-defibrillators utilize the adapters to receive and analyze the vibration-based data in emergency settings. In particular cases, the monitor-defibrillators utilize the signals received via the adapters to provide rapid and accurate responses to complex and/or unexpected subject conditions with which the vibration is associated. In some examples, rapid and accurate responses enabled via the routing, by the adapters, of the signals with the vibration-based data, increase likelihoods of successful administration of medical treatment to subjects. For instance, the adapters enable the monitor-defibrillators to utilize the data received from the wearable devices to identify one or more breath parameters, one or more heartbeat parameters, one or more NIBP parameters, and/or one or more gastrointestinal parameters. In some examples, individual ones of the parameter(s) are associated with, and/or indicate a value representing individual levels of one or more characteristics (e.g., one or more heartbeat characteristics, one or more breath characteristics, one or more NIBP characteristics, and/or one or more gastrointestinal characteristics). In such an instance or another instance, the adapters enable the monitor-defibrillators to output information corresponding to the breath parameters(s), the heartbeat parameters(s), the NIBP parameters(s), and/or the gastrointestinal parameters(s) to increase likelihoods of saving subject lives.

In various cases, the adapters enable the monitor-defibrillators to identify whether any artifacts (e.g., noises) overlap with activity associated with the characteristic(s) (e.g., activity occurring during the generating of the vibration data). For instance, the adapters enable the monitor-defibrillators to identify and/or analyze the vibration data in response to identifying there are no artifacts that may otherwise obscure the vibration data and/or the characteristic(s) (e.g., the parameter(s)) identified therewith.

Various examples will now be described with reference to the accompanying drawings.

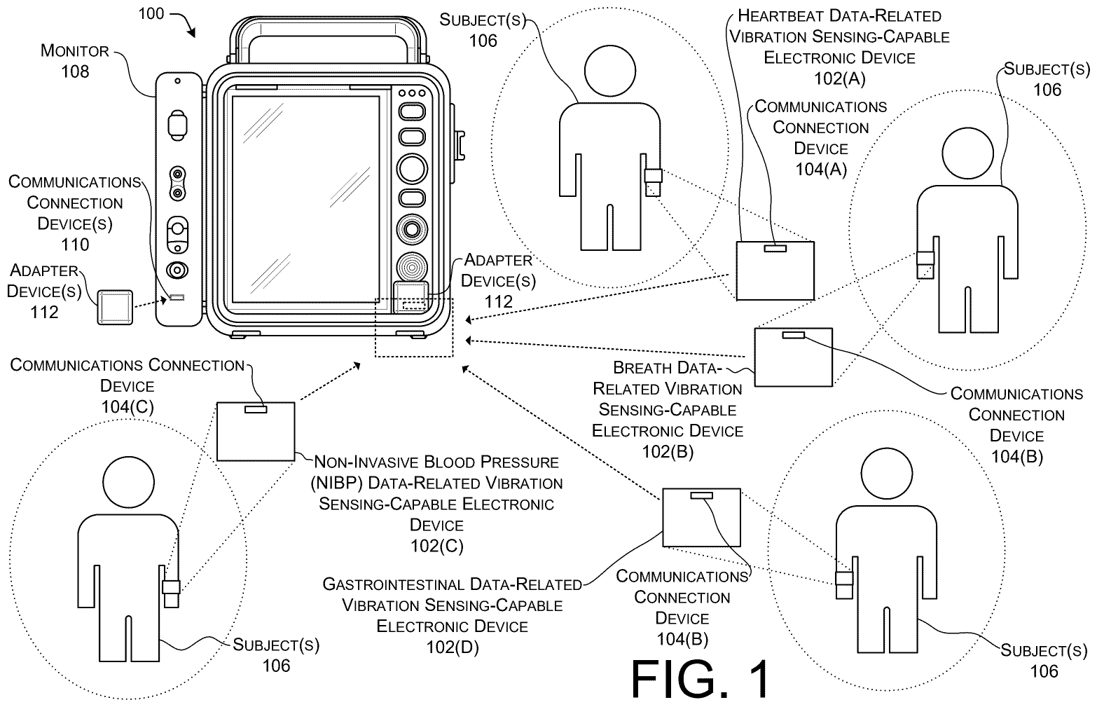

FIG. 1 illustrates an example environment 100 in which vibration-sensing devices are configured to sense vibration and communicate signals via adapters that convert and route the signals to medical devices, the signals including different types of sensor data generated based on the vibration.

In various implementations, the environment 100 includes a heartbeat data-related vibration sensing-capable electronic device (also referred to herein simply as “heartbeat vibration-sensing device”) 102(A), a breath data-related vibration sensing-capable electronic device (also referred to herein simply as “breath vibration-sensing device”) 102(B), a NIBP data-related vibration sensing-capable electronic device (also referred to herein simply as “NIBP vibration-sensing device”) 102(C), and a gastrointestinal data-related vibration sensing-capable electronic device (also referred to herein simply as “gastrointestinal vibration-sensing device”) 102(D), also collectively referred to herein as vibration-sensing devices 102. Examples of the vibration-sensing devices 102 include, for instance, wearable devices (e.g., wrist-worn devices, such as smart watches), mobile devices (e.g., smart phones), wearable medical devices (e.g., wearable defibrillators), implantable devices (e.g., implantable pacemakers), and the like.

In some examples, various types of vibration-based data are managed by the vibration-sensing devices 102. For instance, the vibration-sensing devices 102 are configured to detect vibration. In addition, the vibration-sensing devices 102 are configured to generate data indicative of the vibration (also referred to as “vibration-based data”).

In various examples, the environment 100 includes communications connection devices 104, which are included in the vibration-sensing devices 102. For example, the heartbeat device 102(A), the breath device 102(B), the NIBP device 102(C), and the gastrointestinal device 102(D) include communications connection devices 104(A)-104(D), respectively, also collectively referred to herein as the communications connection devices 104.

In some examples, individual ones of the communications connection devices 104 include any of various types of devices. For examples, any of the communications connection devices 104 includes a transceiver (e.g., an antenna), port, connector, or the like utilized to exchange (e.g., with the monitor 108) any of the signal(s).

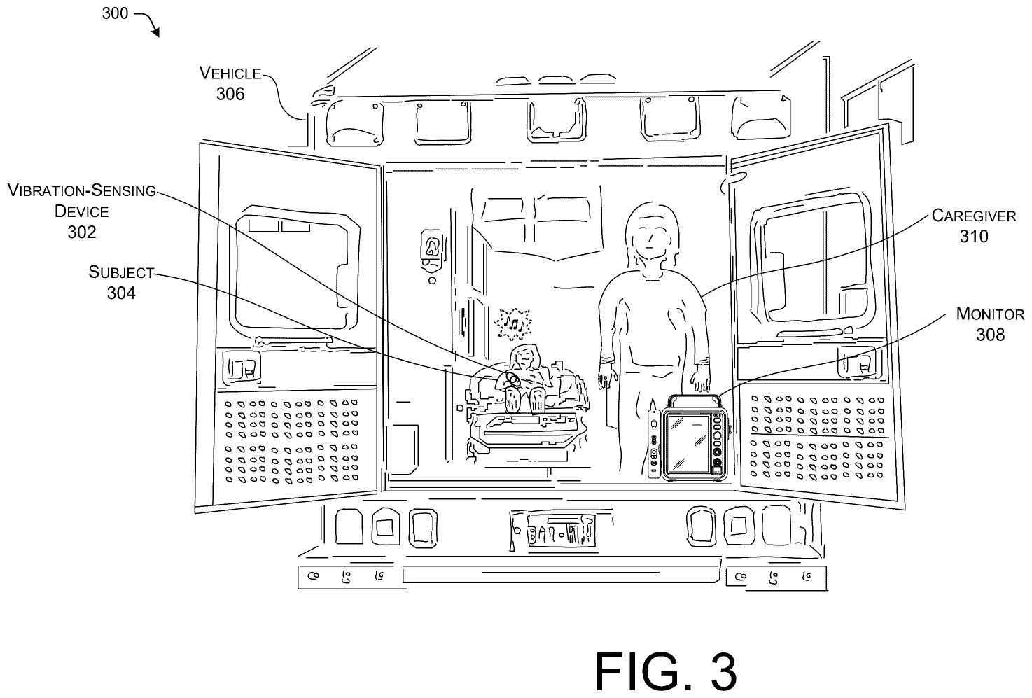

The vibration-sensing devices 102 are configured to monitor one or more subjects 106. For example, the subject(s) 106 include a person undergoing a medical emergency, such as cardiac arrest. In various cases, the vibration-sensing devices 102 include sensors configured to detect a movement and/or sound (also referred to as a “vibration”) of the subject(s) 106. For instance, the vibration-sensing device(s) 102 include one or more accelerometers, gyroscopes, or other movement sensors configured to detect a movement of one or more portions of the subject(s) 106. In some examples, the vibration-sensing device(s) 102 include one or more microphones or piezoelectric sensors configured to detect pressure waves originating from the subject(s) 106 and/or traveling through the subject(s) 106. In some examples, the vibration-sensing devices 102 are configured to manage (e.g., identify, determine, generate, obtain, transmit, delete, modify, analyze, present, retrieve, replace, divide, collate, distribute, etc., or any combination thereof) various types of vibration data. The term “vibration data,” for instance, refers to any data that is indicative of a vibration sensed by the vibration-sensing devices 102. In some cases, the vibration data includes sound data, as discussed below in further detail with reference to FIG. 3.

Various types of vibration data are generated by the vibration-sensing devices 102. For instance, the heartbeat device 102(A) is configured to detect a vibration associated with the movement of blood through the circulatory system of the subject(s) 106. In some cases, the heartbeat device 102(A) detects a vibration of the chest of the subject(s) 106 every time the atria and/or ventricles of the subject(s) 106 contracts or expands. In some cases, the vibration is detected based on the flow of blood through one or more blood vessels through the subject(s) 106. Thus, the heartbeat device 102(A) generates vibration data that is indicative of a heartbeat of the subject(s) 106.

In some cases, the breath device 102(B) is configured to detect a vibration associated with the movement of air through the subject(s) 106 and/or the movement of at least a portion of the respiratory system of the subject(s) 106. For example, the breath device(s) detects a vibration indicative of each time the subject(s) 106 spontaneously breathes or is administered with positive pressure ventilation. Thus, the breath device 102(B) is configured to generate vibration data that is indicative of spontaneous or artificial respiration of the subject(s) 106.

In some examples, the NIBP device 102(c) is configured to detect a vibration associated with a blood pressure measurement administered to the subject(s) 106. For instance, the NIBP device 102(c) includes a blood pressure cuff configured to constrict around an extremity (e.g., arm) of the subject(s) 106 to restrict flow of blood through one or more blood vessels in the extremity. The NIBP device 102(c) is further configured to detect a vibration indicative of blood pumping through an artery in the extremity when the blood pressure cuff is deflated. In various cases, the NIBP device 102(c) is further configured to detect a systolic and/or diastolic pressure of the subject(s) 106 based on the timing of the blood pumping through the artery. In various cases, the NIBP device 102(c) is configured to generate vibration data that is indicative of the movement of blood through one or more arteries of the subject(s) 106.

In various cases, the gastrointestinal device 102(D) is configured to detect a vibration associated with gastric motility, movement of a fluid (e.g., a gas), or some other characteristic of gastrointestinal function of the subject(s) 106. In various cases, the gastrointestinal device 102(D) is configured to generate vibration data that is indicative of the gastrointestinal function of the subject(s) 106.

In various implementations, the vibration-sensing devices 102 are configured to communicate the vibration data to a monitor 108. For example, individual ones of the communication connection devices 104 are communicatively with the monitor 108. In such an example or another example, individual ones of the vibration-sensing devices 102 are, via the communication connection devices 104, in communication with the monitor 108. In various cases, the monitor 108 is utilized to monitor vibration-based data associated with the subject(s) 106.

In various examples, the environment 100 includes one or more communications connection devices 110 in the monitor 108. In some cases, individual ones of the communications connection device(s) 110 include a port (e.g., a universal serial bus (USB) port, as discussed below in further detail). In various cases, the communications connection device(s) 110 includes, as the port, a receptacle, such as a female connector.

In various implementations, the environment 100 includes one or more adapter devices (also referred to herein simply as “adapter(s)”) 112. In some cases, individual ones of the adapter(s) 112 include a communications connection device, such as a connector (e.g., a prong). In various cases, the communications connection device(s) of the adapter(s) 112 includes, as the connector, a male connector, such as a plug (e.g., a USB plug, as discussed below in further detail). In some examples, the port(s) is compatible with the communications connection device(s) 110 (e.g., the receptacle(s) of the monitor 108). For instance, the communications connection device(s) of the adapter(s) 112 is configured to be inserted in the communications connection device(s) 110 (e.g., in any of the port(s) of the communications connection device(s) 110).

In various implementations, the vibration-sensing devices 102 are communicatively coupled to the monitor 108 via the communications connection device(s) 110, which are physically connected to the adapter(s) 112. For instance, the communications connection device(s) 110 are physically connected to the connector(s) in the adapter(s) 112. In some examples, individual ones of the vibration-sensing devices 102 are interconnected with the monitor 108, such as the medical device. In those or other examples, individual ones of the communications connection devices 104 are interconnected with the communications connection device(s) 110, via the adapter(s) 112.

In some examples, the adapter(s) 112 enable the monitor 108 to utilize the data received from the vibration-sensing devices 102 to (e.g., identify, determine, generate, obtain, transmit, delete, modify, analyze, present, retrieve, replace, divide, collate, distribute, etc., or any combination thereof) the one or more breath parameters, one or more the heartbeat parameters, one or more the NIBP parameters, and/or one or more the gastrointestinal parameters. In some examples, individual ones of the parameter(s) are associated with, and/or indicate a value representing a level of any of one or more characteristics. For instance, the characteristic(s) include one or more heartbeat characteristics, one or more breath characteristics, one or more NIBP characteristics, and/or one or more gastrointestinal characteristics. In such an instance or another instance, the adapter(s) 112 enable the monitor 108 to manage information corresponding to the parameters(s) to increase likelihoods of saving subject lives.

In various implementations, individual ones of the vibration-sensing devices 102 are operated in one or more states (e.g., vibration-based data type modes) corresponding to the type(s) of the vibration-based data being generated and/or transmitted. For instance, any individual one of the vibration-sensing devices 102 is utilized to selectively operate in a mode corresponding to managing one of the vibration-based heartbeat data, the vibration-based breath data, the vibration-based NIBP data, or the vibration-based gastrointestinal data. In some examples, the vibration-sensing device 102 switches between any of the modes. In some examples, the switching to a mode, and/or changing to a different mode, is in response to a selection received via user input to a user interface (UI) of the vibration-sensing device 102.

In some cases, the vibration-sensing device 102 operating in any of the vibration-based data type mode(s) supplies power to components utilized to receive the selection(s) via user input to the UI of the vibration-sensing device 102. Alternatively, the vibration-sensing device 102 operating in any of the vibration-based data type mode(s) does not supply power to any component (e.g., the input device) utilized to receive the selection(s).

In some examples, the vibration-sensing device 102 operating in any of the vibration-based data type mode(s) supplies power to components (e.g., a sensor) utilized to generate a type of the vibration-based data to which a mode corresponds. In those or other examples, the vibration-sensing device 102 operating in a vibration-based data type mode does not supply power to components (e.g., sensors) utilized to generate other types of the vibration-based data. For instance, the vibration-sensing device 102 operating in the vibration-based data type mode utilized to manage the vibration-based heartbeat data does supplies power to components (e.g., a sensor) utilized to generate the vibration-based heartbeat data. In such an instance or another instance, the vibration-sensing device 102 operating in a vibration-based data type mode utilized to manage the vibration-based heartbeat data does not supply power to components utilized to generate other types of the vibration-based data (e.g., the vibration-based breath data, the vibration-based NIBP data, or the vibration-based gastrointestinal data). In some examples, power is supplied or not supplied to components for the vibration-based data type mode utilized to manage any other type of vibration-based data in a similar way as for the vibration-based data type mode utilized to manage the vibration-based heartbeat data.

In some cases, individual ones of the vibration-sensing devices 102 are operated in one or more states (or “operating state(s)”) corresponding to one or more types of operations. For instance, the state(s) include a dormant state, a monitor state, and a communicate (e.g., transmit) state. In some examples, the switching to a state, and/or changing to a different state, is in response to a selection received via user input to the vibration-sensing device 102.

In some examples, a vibration-sensing device 102 operating in the dormant state receives signals utilized to change between the state(s) and/or identifies any of one or more triggers utilized to change between the state(s). In those or other examples, the vibration-sensing device 102 operating in the dormant state does not monitor (e.g., sense) vibration or transmit data (e.g., any of the vibration-based data). In those or other examples, the vibration-sensing device 102 operating in the dormant state does not supply power to any components utilized to monitor (e.g., sense) vibration or transmit data. In those or other examples, the vibration-sensing device 102 operating in the dormant state does not supply power to any sensor and/or any transceiver.

In some examples, the vibration-sensing device 102 operating in the dormant state does not supply power to any of the components of the vibration-sensing device 102. For instance, the vibration-sensing device 102 operating in the dormant state receives a signal (e.g., from the monitor 108) and changes to another state. In some cases, the vibration-sensing device 102 changes to the other state based on the signal powering a switch utilized to supply power from a power supply of the vibration-sensing device 102 to the processor of the vibration-sensing device 102. In various cases, the vibration-sensing device 102 supplies power to the processor of the vibration-sensing device 102 in response to receiving the signal inducing a current in a circuit that passively activates the switch. In some examples, the vibration-sensing device 102 supplies power to the processor of the vibration-sensing device 102 in response to receiving the signal inducing the current activating the switch that connects the power supply to the processor. In those or other examples, the processor of the vibration-sensing device 102 receiving the power controls the vibration-sensing device 102 the vibration-sensing device 102 to switch from the dormant state to the monitor state or the transmit state.

In various examples, such as with a vibration-sensing device 102 in the dormant state being enabled in the dormant state to supply power to the processor, the vibration-sensing device 102 receives a signal (e.g., from the monitor 108) and changes to another state. For instance, the processor of the vibration-sensing device 102 identifies the signal being received (e.g., from the monitor 108) and changes to another state in response to the identifying of the signal. In such an instance, the processor of the vibration-sensing device 102 identifies that the signal a trigger to switch states, and changes to another state (e.g., a state identified in the signal).

In some cases, the trigger(s) include various types of time-based triggers. For instance, the trigger(s) include a trigger that is set periodically, a trigger that is set at a specific time, and/or a trigger that is set after a time period expires from when the vibration-sensing device 102 begins operating in a corresponding state. In some cases, any or all of the trigger(s) utilized to control a vibration-sensing device 102 to operate in any of the state(s) is the same as, or different from, any or all of the trigger(s) utilized to control the vibration-sensing device 102 to operate in any other state.

In some examples, a vibration-sensing device 102 operating in the monitor state monitors (e.g., senses) vibration, and/or is enabled to monitor (e.g., sense) vibration. In those or other examples, the vibration-sensing device 102 operating in the monitor state does not supply power to any components utilized to transmit data. In those or other examples, the vibration-sensing device 102 operating in the monitor state does not supply power to any transceiver. In various cases, the vibration-sensing device 102 operating in the monitor state receives signals utilized to change between the state(s) and/or identifies any of one or more triggers utilized to change between the state(s), as in the dormant state. Alternatively, the vibration-sensing device 102 operating in the monitor state does not receive signals utilized to change between the state(s) and/or identify any of trigger(s) utilized to change between the state(s).

In some examples, a vibration-sensing device 102 operating in the transmit state transmits data (e.g., vibration-based data), and/or is enabled to transmit data. In those or other examples, the vibration-sensing device 102 operating in the transmit state does not supply power to any components utilized to monitor data. In those or other examples, the vibration-sensing device 102 operating in the transmit state does not supply power to any sensor. In various cases, the vibration-sensing device 102 operating in the transmit state receives signals utilized to change between the state(s) and/or identifies any of one or more triggers utilized to change between the state(s), as in the dormant state. Alternatively, the vibration-sensing device 102 operating in the transmit state does not receive signals utilized to change between the state(s) and/or identify any of trigger(s) utilized to change between the state(s).

In some examples, any of the vibration-sensing devices 102 supplies power to components utilized to operate in any of the operating state(s), but not to remaining components. In some examples, a vibration-sensing device 102 operating in the dormant state supplies power to a transceiver and the processor to receive signals utilized to change between the state(s) and/or identify any of the trigger(s). In those or other examples, the vibration-sensing device 102 operating in the dormant state does not supply power to any other components of the vibration-sensing device 102. Alternatively, the vibration-sensing device 102 operating in the dormant state supplies power to the processor to identify any of the trigger(s), but not to any other components (e.g., such as any of the transceivers) of the vibration-sensing device 102. In some cases, the transceiver receiving signals utilized to change between the state(s) is different from the transceiver utilized to exchange other signals, such as signals for communicating the vibration-based data. Alternatively, the transceiver receiving signals utilized to change between the state(s) is also utilized to exchange other signals, such as signals for communicating the vibration-based data.

In some cases, the vibration-sensing device 102 operating in any of the operating state(s) supplies power to components utilized to receive the selection(s) via user input to the UI of the vibration-sensing device 102. Alternatively, the vibration-sensing device 102 operating in any of the operating state(s) does not supply power to any component (e.g., the input device) utilized to receive the selection(s). In various examples, the vibration-sensing device 102 operating in the monitor state and the transmit state, but not the dormant state, supplies power to any component utilized to receive the selection(s). Alternatively, the vibration-sensing device 102 operating in the transmit state, but not the dormant state or the monitor state, supplies power to any component utilized to receive the selection(s).

In various implementations, any combination of the vibration-sensing devices 102 is integrated together as a single one of the vibration-sensing devices 102. For instance, any of the vibration-sensing devices 102 is utilized to manage any types of the vibration-based data. In such an instance or another instance, any individual one of the vibration-sensing devices 102 is utilized to manage the vibration-based heartbeat data, the vibration-based breath data, the vibration-based NIBP data, and/or the vibration-based gastrointestinal data.

In various implementations, any of the vibration-sensing devices 102 is of a different type from remaining ones of the vibration-sensing devices 102. For instance, any of the vibration-sensing devices 102 is utilized to manage a single type of the vibration-based data. In such an instance or another instance, any individual one of the vibration-sensing devices 102 is utilized to independently manage the vibration-based heartbeat data, the vibration-based breath data, the vibration-based NIBP data, or the vibration-based gastrointestinal data.

In various implementations, the monitor 108 is operated in one or more modes (e.g., vibration-based data type modes) associated with the type(s) of the vibration-based data being received. For instance, the monitor 108 is utilized to selectively operate in a mode corresponding to managing one of the vibration-based heartbeat data, the vibration-based breath data, the vibration-based NIBP data, or the vibration-based gastrointestinal data. In various cases, the monitor 108 is utilized to selectively operate in a mode corresponding to managing a combination of two or more of the vibration-based heartbeat data, the vibration-based breath data, the vibration-based NIBP data, and the vibration-based gastrointestinal data. In some examples, the monitor 108 switches between any of the modes. In some examples, the switching to a mode, and/or changing to a different mode, Is in response to a selection received via user input to an UI of the monitor 108.

In some examples, the monitor 108 automatically operates in any of the mode(s) in response to receiving a corresponding type of the vibration-based data. In those or other examples, the monitor 108, currently operating in a mode, automatically operates in a combination of the current mode and a different mode in response to receiving a different type of the vibration-based data. Alternatively, the monitor 108, currently operating in a mode, automatically stops operating in the current mode, and switches to operating in a different mode in response to receiving the different type of the vibration-based data. Alternatively, the monitor 108, currently operating in a combination of modes, automatically stops operating in the current combination of modes, and switches to operating in a different mode in response to receiving the different type of the vibration-based data. Alternatively, the monitor 108, currently operating in a mode, automatically stops operating in the current mode, and switches to operating in a combination of different modes in response to receiving the combination of different types of the vibration-based data.

In various cases, the monitor 108 identifies whether to enable operation of a mode in combination wither any other mode based on a priority of the type of vibration-based data with which the mode is associated. For instance, the vibration-sensing device 102 identifies to operate in a mode associated with a type of vibration-based data with a relatively higher priority, by refraining from operating in a combination of the mode with any other mode. In some cases, the monitor 108 identifies to operate in a mode associated with a type of vibration-based data with a relatively lower priority, by operating in a combination of the mode with at least one of the other modes. In some cases, any priority associated with any mode is pre-programmed into the monitor 108. In those or other examples, any priority associated with any mode is able to be set and/or modified by one or more selections via user input to the UI of the monitor 108. Alternatively, any priority associated with any mode is pre-programmed into the monitor 108 and blocked, by the monitor 108, from being modified by any of the selection(s) received via user input.

In some examples, the vibration-sensing device 102 operates in any of the state(s) in combination with any of the mode(s). In those or other examples, the vibration-sensing device 102 begins, continues, discontinues, ceases, etc., operating in any of the state(s) and/or any of the mode(s). For instance, the vibration-sensing device 102 automatically selects any of the state(s) and/or the mode(s). In some cases, the vibration-sensing device 102 selects any of the state(s) and/or the mode(s) in response to receiving one or more selections via user input to the UI of the vibration-sensing device 102. In various examples, the vibration-sensing device 102 selects any of the state(s) and/or the mode(s) in response to receiving one or more signals from the monitor 108. In alternative examples, the vibration-sensing device 102 overrides (e.g., via override settings stored in the vibration-sensing device 102 by selection(s) via user input, and/or via override settings stored in the vibration-sensing device 102 by communications from the monitor 108) signal(s) signals for certain types of state(s) and/or certain types of mode(s) notwithstanding the signals being received from the monitor 108.

In various implementations, any of the vibration-sensing devices 102 includes any of various types of devices, such as one or more medical devices, one or more non-medical devices, or any combination thereof. For instance, any of the vibration-sensing devices 102 (e.g., including the medical device) includes a sensor, a wireless sensor, a modular sensor, a peripheral, a wireless peripheral, a modular peripheral, a cable, a wireless cable, a modular cable, a connector, a wireless connector, a modular connector, a lead, a wireless lead, a modular lead, any other type of medical device, or any combination thereof.

In some cases, any of the vibration-sensing devices 102 (e.g., including the medical device) includes a medical sensor. For instance, any of the vibration-sensing devices 102 (e.g., including the medical sensor) includes a physiological data device (e.g., one or more physiological data sensors), a CPR feedback device (e.g., one or more CPR feedback sensors), any other type of medical sensor, or any combination thereof.

For instance, any of the vibration-sensing devices 102 (e.g., including the medical device) includes a physiological data sensor, the CPR feedback device. In various examples, the CPR feedback device includes sensors (e.g., accelerometers) and measures, via the sensors, a depth of a chest of the subject(s) 106 while a caregiver is administering CPR. In various cases, the CPR feedback devices provides feedback (e.g., real time feedback), such as feedback being provided dynamically, on a quality of the CPR being administered. In some instances, the CPR feedback device includes a “puck” capable of being placed on the subject(s) 106.

In other implementations, any of the vibration-sensing devices 102 includes the non-medical device. For instance, any of the vibration-sensing devices 102 (e.g., including the non-medical device) includes a sensor, a wireless sensor, a modular sensor, a peripheral, a wireless peripheral, a modular peripheral, a cable, a wireless cable, a modular cable, a connector, a wireless connector, a modular connector, a lead, a wireless lead, a modular lead, any other type of non-medical device, or any combination thereof.

In some cases, any of the vibration-sensing devices 102 (e.g., including the non-medical device) includes a non-medical sensor. For instance, any of the vibration-sensing devices 102, (e.g., including the non-medical sensor) includes an asset tracking sensor associated with an asset (e.g., item), a product sensor associated with a product, a package sensor associated with a package, a warehouse sensor associate with a warehouse (e.g., a location in a warehouse), a retail sensor, a logistical sensor, a badge sensor (e.g., an employee badge sensor), a location sensor (e.g., a global positioning system (GPS) sensor), a payment sensor associated with a payment (e.g., a payment transaction) of an item, a purchase sensor associated with a purchase of an item, a price sensor associated with a price of an item, a tracking sensor, an item identifier sensor associated with an item (e.g., an object, any other type of item, or any combination thereof), a user identifier sensor associated with a user (e.g., a person, an employee, a subject, a caregiver, any other type of user, or any combination thereof), a network identifier sensor associated with a network, a network adapter identifier sensor (e.g., a cellular network adapter identifier sensor, a wi-fi network adapter identifier sensor, any other type of network adapter identifier sensor associated with any other type of network, or any combination thereof), a network identifier sensor (e.g., a cellular network identifier sensor, a wi-fi network identifier sensor, any other type of network identifier sensor associated with any other type of network, or any combination thereof), a device sensor (e.g., a vibration-sensing device sensor, an internet of things (IoT) device sensor, a computing sensor, a desktop computer sensor, a laptop sensor, a tablet sensor, a personal digital assistant (PDA) sensor, an electronic book device sensor, a server sensor, a workstation sensor, an audio device sensor, or any other device sensor associated with any other type of device, or any combination thereof), a phone sensor (e.g., a cellular phone sensor) associated with a phone, a camera sensor associated with a camera, an instrument sensor (e.g., a field-device sensor, an electronic instrument sensor, any other type of instrument sensor, or any combination thereof, an electronic stereo sensor, an audio instrument sensor, an electronic speaker sensor, any other type of instrument sensor associated with any other type of instrument, or any combination thereof), a vehicle sensor associated with a vehicle, a parking sensor associated with a parking location, any other type of non-medical sensor, or any combination thereof.

In various examples, the vibration sensor(s) of any of the vibration-sensing devices 102 is utilized to generate the vibration data, being associated with one or more physiological conditions of the subject(s) 106. In such an instance or another instance, the physiological condition(s) with which the vibration data is associated include one or more heartbeat conditions, one or more breath conditions, one or more NIBP conditions, and/or one or more gastrointestinal conditions. In various cases, the physiological condition(s) with which the vibration data is associated (e.g., utilized to identify) includes, with respect to a subject 106, a heart murmur, fluid in one or more lungs, etc., or any combination thereof.

In various cases, individual ones of the vibration-sensing devices 102 and/or the monitor 108 are utilized to identify whether one or more artifacts exist (e.g., occur and/or are present during sensing of the vibration). In some examples, in response to identifying the artifact(s) exist, the vibration-sensing device 102 and/or the monitor 108 perform any of one or more actions. In some cases, the action(s) includes initiating one or more alarms. For instance, the action(s) include, possibly in response to the initiating of the alarm(s), generating the alarm(s). In such an instance or another instance, the initiating of (e.g., setting one or more triggers for) the alarm(s) and the generating of (e.g., causing output of) the alarm(s) are integrated together and performed as a single action. In some examples, the alarm(s) include any of various types of alarms, being visual, audible, haptic, etc., or any combination thereof.

In those or other examples, in response to identifying the artifact(s) exist, the vibration-sensing device 102 and/or the monitor 108 waits to identify, generate, analyzed, etc., or any combination thereof, the vibration data. In some instances, identifying the artifact(s) exist enables the vibration-sensing device 102 and/or the monitor 108 to perform the action(s) in response to identifying the artifact(s). In those or other instances, identifying the artifact(s) exist enables the vibration-sensing device 102 and/or the monitor 108 to perform the action(s) in response to identifying that, due to the artifact(s), the vibration data is skewed, unreliable, inaccurate, weak, misreprentative, etc., or any combination thereof, of the vibration (e.g., with which the vibration data is associated).

In various cases, the vibration-sensing device 102 and/or the monitor 108 identify that there are no artifacts (e.g., no interfering/disrupting sounds) occurring during activity associated with vibration. For example, in response to identifying, by the vibration-sensing device 102 and/or the monitor 108, that there are no artifacts (e.g., interfering with sensing of the vibration-sensing device 102), the vibration-sensing device 102 and/or the monitor 108 proceed with identifying the vibration data and/or the vibration characteristic(s) (e.g., the parameter(s)).

In alternative or additional examples, for instance with the vibration-sensing devices 102 and/or the monitor 108 identifying that the artifact(s) exists, the vibration-sensing devices 102 and/or the monitor 108 filter out any of one or more portions of data that include, and/or are affected by, the artifact(s). For instance, the vibration-sensing devices 102 and/or the monitor 108 filter out, from data that includes the vibration data, the portion(s) of data that includes, and/or that is affected by, the artifact(s). In such an instance or another instance, the vibration-sensing device 102 and/or the monitor 108 proceed with identifying the vibration data and/or the vibration characteristic(s) (e.g., the parameter(s)), in a similar way as discussed above for when no artifact(s) exist. For example, any noise that obscures the vibration data is excluded from analysis, by the vibration-sensing devices 102 and/or the monitor 108, of the vibration data.

In alternative or additional examples, for instance with the vibration-sensing devices 102 and/or the monitor 108 identifying that the artifact(s) exists, the vibration-sensing devices 102 and/or the monitor 108 cancel noise associated with (e.g., resulting from) the artifact(s). For instance, the vibration-sensing devices 102 and/or the monitor 108 perform noise cancellation, by removing noise from data that includes the vibration data. In such an instance or another instance, the vibration-sensing device 102 and/or the monitor 108 proceed with identifying the vibration data and/or the vibration characteristic(s) (e.g., the parameter(s)), in a similar way as discussed above for when no artifact(s) exist. For example, the vibration-sensing device 102 and/or the monitor 108 filter out, from the data generated by the vibration, noise identified as being unrelated to a characteristic (e.g., a heartbeat) of the subject 106.

In various examples, the monitor 108 detects a signature routed from the vibration-sensing device 102 and by the adapter(s) 112, in response to identifying the adapter(s) 112 being inserted in the communications connection device(s) 110 and being activated by power supplied from by the monitor 108. In some cases, the monitor 108 receives data from the vibration-sensing device 102 via the communications connection device(s) 110 in response to the identifying that the vibration-sensing device 102 is activated to detect a vibration corresponding to a breath of a subject 106 on which the vibration-sensing device 102 has been placed.

In various implementations, the monitor 108 identifies a pattern of the data that matches, at a level that is greater than a threshold level, another pattern of other data (e.g., stored in the monitor 108 and/or one or more remote devices, in communication with the monitor 108). For instance, the other pattern of the other data being associated with activity overlapping with an artifact. In various cases, the monitor 108, in response to the identifying of the pattern of the data matches the other pattern of the other data, identifies that activity associated with the pattern of the data overlaps with the artifact. In various examples, the monitor 108, in response to the identifying that the pattern of the data overlaps with the artifact, generates an alarm advising a rescuer to treat a corresponding condition (e.g., associated with a heartbeat, a breath, an NIBP, and/or a gastrointestinal characteristic) of the subject 106.

In some examples, the monitor 108 identifies a pattern (e.g., a new pattern) of data (e.g., new data); and identifies the new pattern of the new data is associated with an ECG parameter. In those or other examples, the monitor 108 identifies the new pattern of the new data is associated with a characteristic (e.g., a parameter).

In various cases, the monitor 108 identifies a likelihood that the vibration data matches stored data representing a historical vibration (e.g., stored in the monitor 108 and/or the remote device(s)) that overlaps with an artifact, and that the likelihood is above a threshold likelihood. In some examples, the monitor 108, in response to identifying that the likelihood is above the threshold likelihood, determines that the characteristic and/or activity (e.g., associated with the heartbeat, the breath, the NIBP, the gastrointestinal function, or any combination thereof) generating the vibration overlaps with the artifact. In some cases, the monitor 108, in response to determining that the characteristic and/or the activity generating the vibration overlaps with the artifact, generates an alarm, and outputs the alarm.

In various cases, the monitor 108 receives, via the adapter(s) 112 and using a communication protocol (e.g., a wireless protocol), other data representing another vibration associated with another characteristic and/or another activity (e.g., associated with the heartbeat, the breath, the NIBP, the gastrointestinal function, or any combination thereof). In some examples, the monitor 108 analyzes the other data being converted by the adapter(s) 112 to other converted data associated with the different protocol (e.g., a wired protocol). In those or other examples, the monitor 108 identifies another likelihood that the other data matches other stored data representing another historical vibration associated with a historical characteristic (e.g., associated with the heartbeat, the breath, the NIBP, the gastrointestinal function, or any combination thereof) that overlaps with another artifact, and that the other likelihood is above another threshold likelihood. The historical vibration is stored, for instance, in the monitor 108 and/or the remote device(s). In those or other examples, the monitor 108, in response to identifying that the other likelihood is above the other threshold likelihood, determines that the activity associated with the characteristic overlaps with the other artifact.

In some examples, the vibration-sensing devices 102 includes an activator configured to trigger the sensor to detect sound via the vibration. For example, the activator is configured to trigger a transceiver of the monitor 108 to transmit the communication signal. In various cases, the monitor 108 receives, from the vibration-sensing devices 102 and via the adapter(s) 112, a tag representing that the vibration-sensing devices 102 has a capability to sense the vibration. In some examples, the monitor 108 transmits a prompt to the vibration-sensing devices 102 that triggers the vibration-sensing devices 102 to transmit the data to the monitor 108. In various cases, the monitor 108 receives the data, in response to the transmitting of the prompt. For instance, the prompt activates a state (e.g., a communicating state) of the monitor 108 to receive the data, and the monitor 108 in the communicating (e.g., receiving) state, receives the data.

In various examples, the vibration-sensing devices 102 and the monitor 108 exchange one or more codes prior to communicating. For instance, the monitor 108 exchanges the code(s) utilized to pair the monitor 108 with the vibration-sensing devices 102 via the connector. In some cases, the monitor 108, in response to the exchanging of the codes, receives the data representing the vibration (e.g., generated by the heart emitting the heartbeat, or by any other of the characteristic(s)). In some cases, the monitor 108 identifies a score that corresponds to an accuracy of a characteristic (e.g., of the heartbeat) identified utilizing the data, and that the score is less than a threshold score. For instance, the monitor 108, in response to the identifying that the score is less than the threshold score, transmits a pulse triggering a change in a state of a vibration-sensing device 102 to another state (e.g., a transmit state) of the vibration-sensing device 102 that increases an amount of power drawn from a power source by the vibration-sensing device 102. In some cases, the monitor 108 receives data from the vibration-sensing device 102 operating in the other state.

In some cases, the monitor 108 identifies a score analogous to an accuracy with which a characteristic of a pattern (e.g., of the heartbeat) is aligned with another characteristic of another pattern. For instance, the other characteristic of the other pattern is previously generated by activity that is physical or by activity that is electrical. In some cases, the activity that is physical is sensed by a sensor (e.g., an accelerometer) that is attached to the subject 106. In some cases, the activity that is electrical being sensed via a sensor placed against a body of the subject 106.

In various examples, the monitor 108 monitors whether a characteristic (e.g., of a heartbeat or other type characteristic) exceeds a threshold, or whether the characteristic is indeterminate or unclassifiable. For instance, the monitor 108, in response to the monitoring, transmits a trigger causing the vibration-sensing device 102 to retransmit data generated by other vibration occurring subsequently to the vibration. In some cases, the monitor 108 identifying a weight associated with a treatment representing a likelihood that the treatment will be successful in improving a condition of the subject 106. For instance, the monitor 108, in response to identifying the weight, generates an advisory instructing a rescuer to provide treatment to the subject 106.

In some cases, the monitor 108 maps the data to individual occurrences in historical data (e.g., stored in the monitor 108 and/or the remote device(s)) identifying patterns of corresponding heartbeats. In various examples, the monitor 108 identifies that a likelihood of a match between the data and an occurrence in the historical data is above a threshold. For instance, the monitor 108 identifies that the occurrence in the historical data does not include any unexpected patterns; and refrains from generating an advisory representing a characteristic (e.g., a heartbeat) of the subject 106 in response to the identifying that the occurrence in the historical data does not include any unexpected patterns.

In various cases, the monitor 108, in response to identifying the artifact, identifies a classification associated with a historical artifact (e.g., stored in the monitor 108 and/or the remote device(s)) matching the artifact by a score being above a threshold score. In some examples, the monitor 108, in response to the identifying of the classification associated with the historical artifact, identifies whether the classification is associated with the artifact. For instance, the monitor 108 identifies an index (e.g., identifier of an alarm type, being visual, audible, haptic, etc., or any combination thereof) representing the alarm, the index being associated with the classification; generates an alarm, and outputs the alarm. For instance, the monitor 108 ceases output of the alarm in response to receiving other data relayed by the adapter(s) 112, the other data representing another vibration associated with a characteristic (e.g., the heartbeat, the breath, etc.) of the subject 106.

In various cases, the monitor 108 outputs a notifier to a rescuer of an availability of the vibration-sensing device 102. For instance, the monitor 108, in response to outputting the notifier and identifying the adapter(s) 112 being inserted into the communications connection device(s) 110, generating, via the adapter(s) 112, the communication path; and cease output of the alarm in response to receiving other data relayed by the adapter(s) 112, the other data representing another vibration associated with the breath of the subject 106.

In various cases, the monitor 108 determines that a characteristic (e.g., the breath) of the subject 106 is indicative of a parameter being below a threshold parameter. For instance, the monitor 108 outputs a notifier (e.g., via the UI of the monitor 108) to a rescuer of an availability of the connector that enables the vibration-sensing device 102 to be utilized as a substitute for a stethoscope. In some cases, the monitor 108, in response to outputting the notifier and identifying the adapter(s) 112 being inserted into the communications connection device(s) 110, generating, via the adapter(s) 112, a communication path between the vibration-sensing device 102 and the monitor 108.

In various cases, the monitor 108 receives the data representing the vibration by detecting, by an activator, a tap of the vibration-sensing device 102 (e.g., to the adapter(s) 112). In some examples, the monitor 108 receives a prompt from the vibration-sensing device 102 being activated by the tap. In those or other examples, the monitor 108 transmitting an acknowledgement to the vibration-sensing device 102; and receives the data in response to receiving the prompt and transmitting the acknowledgement. In various cases, the monitor 108 receives the data representing the vibration generated by a characteristic (e.g., the breath), the receiving of the data ceasing when the adapter(s) 112 is disconnected (e.g., removed) from the port and/or turned off (e.g., via a button on the adapter(s) 112).

In some examples, the vibration-sensing devices 102 includes one or more sensors, such as one or more physiological parameter sensors. For instance, the physiological parameter sensors generated physiological data. In such an instance or another instance, the physiological data includes one or more physiological parameters of the subject(s) 106. Examples of physiological parameters include, for instance, an electrocardiogram (ECG or EKG), an impedance, a force administered to the subject(s) 106, a blood pressure, an airway parameter (e.g., a partial pressure of carbon dioxide, a partial pressure of oxygen, a capnography, an end tidal gas parameter, a flow rate, etc.), a blood oxygenation (e.g., a pulse oximetry value, a regional oximetry value, etc.), an electroencephalogram (EEG), a temperature, a heart sound, a blood flow rate, a physiological geometry (e.g., a shape of a blood vessel, an inner ear shape, etc.), a heart rate, a pulse rate, or the like. For example, the medical sensor in the monitor 108 includes at least one of electrodes, a detection circuit, defibrillator pads (e.g., one or more defibrillator pads receiving power from a power supply, such as an internal power supply, an external power supply, or a combination thereof), a force sensor, a blood pressure cuff, an ultrasound-based blood pressure sensor, an invasive (e.g., intra-arterial) blood pressure sensor (e.g., including a cannula inserted into the subject(s) 106), a gas sensor (e.g., a carbon dioxide and/or oxygen sensor), a flowmeter, a pulse oximetry sensor, a regional oximetry sensor, a thermometer, a microphone, an ultrasound transducer, a medical imaging device (e.g., an ultrasound imaging device), or the like.

In various cases, the monitor 108 manages the vibration-based data utilizing the adapter(s) 112 being interconnected with at least one of the vibration-sensing devices 102. In some examples, managing, by the monitor 108, the vibration-based data includes identifying, determining, obtaining, receiving, deleting, modifying, analyzing, replacing, dividing, collating, distributing, etc., or any combination thereof, the vibration-based data. For instance, the monitor 108 receives the vibration-based data from at least one of the vibration-sensing devices 102. In such an instance and another instance, the vibration-based data is received from at least one of the vibration-sensing devices 102 in response to the generating, by at least one of the vibration-sensing devices 102, of the vibration-based data. In such an instance and another instance, the vibration-based data is received from at least one of the vibration-sensing devices 102 in response to the sensing, by at least one of the vibration-sensing devices 102, of the vibration.

In various implementations, the monitor 108 is includes any of various types of devices, such as one or more other medical devices, one or more other non-medical devices, or any combination thereof. For instance, the monitor 108 (e.g., including the other medical device) includes a monitor device, a defibrillator device (e.g., a monitor-defibrillator or automated external defibrillator (AED)), a mechanical chest compression device, a ventilation device, a subject monitor, a video laryngoscope, a health device, a diagnostic device, a field-deployable medical device, any other type of medical device, or any combination thereof. In other implementations, the monitor 108 includes a non-medical device. For instance, the monitor 108 (e.g., including the other non-medical device) includes a mobile device, a payment device, an inventory device, a record keeping device, an employee badge device, a field-deployable non-medical device, any other type of non-medical device, or any combination thereof.

In some examples, the monitor 108 includes, in the communications connection device(s) 110, a transceiver, such as a wired communication transceiver (or “wired transceiver”). In those or other examples, the wired transceiver included in the communications connection device(s) 110 is compatible with a protocol (e.g., a technology), such as a wired communications protocol (or “wired protocol”). For instance, the wired transceiver included in the communications connection device(s) 110 includes a USB transceiver (e.g., a transceiver compatible with a USB protocol). However, in another instance, the wired transceiver included in the communications connection device(s) 110 includes a recommended standard (RS)-232 transceiver (e.g., a transceiver compatible with an RS-232 protocol), an RS-485 transceiver (e.g., a transceiver compatible with an RS-485 protocol), an ethernet transceiver (e.g., a transceiver compatible with an ethernet protocol), an inter-integrated circuit (I2C) transceiver (e.g., a transceiver compatible with an I2C protocol), a serial parallel interface (SPI) transceiver (e.g., a transceiver compatible with an SPI protocol), a 1-wire transceiver (e.g., a transceiver compatible with a 1-wire protocol), a serial advanced technology attachment (SATA) transceiver (e.g., a transceiver compatible with a SATA protocol), or any other type of wired transceiver (e.g., a transceiver compatible with any other type of wired communication protocol).

In various implementations, the adapter(s) 112 is connected to the monitor 108. In some cases, the adapter(s) 112 being connected to the monitor 108 includes the adapter(s) 112 being physically connected, electrically connected (e.g., connected for exchanging data, power, or any combination thereof), and communicatively connected (e.g., connected for exchanging communication signals (or “signals”)) (e.g., messages), to the monitor 108. For instance, the adapter(s) 112 (e.g., one or more portable adapters) are plugged into the monitor 108, by being inserted into the monitor 108. In such an instance or another instance, the adapter(s) 112 being inserted into the monitor 108 includes the plug of the adapter(s) 112 being inserted into the port of the monitor 108 (e.g., being inserted into the port of the communications connection device(s) 110). In such an instance or another instance, the plug includes of the adapter(s) 112 includes a USB compatible plug (or “USB plug”), which is inserted into the port of the communications connection device(s) 110, the port of the communications connection device(s) 110 including a USB compatible port (or “USB port”).

In various implementations, the adapter(s) 112 (e.g., the communications connection device(s) in adapter(s) 112) is compatible with the communications connection device(s) 110. In some cases, compatibility between the communications connection device(s) 110 and the adapter(s) 112 (e.g., the communications connection device(s) in the adapter(s) 112) includes electrical compatibility, physical compatibility, any other type of compatibility, or any combination thereof. In those or other cases, the compatibility includes the communications connection device(s) 110 and the adapter(s) 112 being connectible, such as electrical connectable, physical connectable, or any combination thereof.

In various examples, the adapter(s) 112 (e.g., the communication connection device(s) in the adapter(s) 112) includes a transceiver, such as another wired transceiver. For instance, the other wired transceiver included in the adapter(s) 112 includes a USB transceiver (e.g., a transceiver compatible with a USB protocol). However, in another instance, the wired transceiver included in the adapter(s) 112 includes an RS-232 transceiver (e.g., a transceiver compatible with an RS-232 protocol), an RS-485 transceiver (e.g., a transceiver compatible with an RS-485 protocol), an ethernet transceiver (e.g., a transceiver compatible with an ethernet protocol), an I2C transceiver (e.g., a transceiver compatible with an I2C protocol), a SPI transceiver (e.g., a transceiver compatible with an SPI protocol), a 1-wire transceiver (e.g., a transceiver compatible with a 1-wire protocol), a SATA transceiver (e.g., a transceiver compatible with a SATA protocol), or any other type of wired transceiver (e.g., a transceiver compatible with any other type of wired communication protocol).

In various examples, the adapter(s) 112 includes a transceiver, such as another wireless transceiver (e.g., another wireless transceiver including one or more other antennas). For instance, the other wireless transceiver included in the adapter(s) 112 includes a short-range communications protocol-capable transceiver. In such an instance or another instance, the other wireless transceiver included in the adapter(s) 112 includes an NFC transceiver (e.g., a transceiver compatible with an NFC protocol). However, in another instance, the other wireless transceiver included in the adapter(s) 112 includes a bluetooth transceiver (e.g., a transceiver compatible with a bluetooth protocol), a BLE transceiver (e.g., a transceiver compatible with a BLE protocol), an ultrasonic transceiver (e.g., a transceiver compatible with a ultrasonic protocol), a zigbee transceiver (e.g., a transceiver compatible with an zigbee protocol), a wi-fi transceiver (e.g., a transceiver compatible with an a wi-fi protocol), or an IEEE 802.15.4 (e.g., a transceiver compatible with an IEEE 802.15.4 protocol) transceiver, a z-wave transceiver (e.g., a transceiver compatible with a z-wave protocol), or any other type of wireless transceiver (e.g., a transceiver compatible with any other type of wireless communication protocol).

In some implementations, the adapter(s) 112 is utilized to communicate (e.g., exchange communications) using signals (e.g., communication signals) that are compatible with various communication protocols, which includes converting the signals (e.g., with the vibration-based data) that are compatible with the various communication protocols. In various examples, the communication protocols with which the adapter(s) 112 is compatible include the wired communication protocol and the wireless communication protocol. For instance, the adapter(s) 112 is compatible with the USB protocol, via the USB transceiver in the adapter(s) 112, and the NFC protocol, via the NFC transceiver in the adapter(s) 112.

In some cases, by utilizing the adapter(s) 112 to translate between the wired protocol utilized by the monitor 108 and the wireless protocol utilized by the vibration-sensing devices 102, important vibration data generated by the vibration-sensing devices 102 is able to be utilized by the monitor 108. In various examples, the monitor 108 being able access, via the translating of protocols, the data generated by the vibration-sensing devices 102 is able to output (e.g., present) crucial information utilized to a caregiver (e.g., a rescuer) to administer aid to a subject 106. For instance, lives of the subject(s) 106 are able to be saved by communicating the vibration data to the monitor 108, which is utilizable by the caregiver (e.g., the rescuer).

In some instances, by utilizing the adapter(s) 112, with the wired transceiver, such as the USB transceiver, and the wireless transceiver, such as the NFC transceiver, the signals with the vibration-based data are exchanged between the vibration-sensing devices 102 and the monitor 108. For example, the signals (e.g., with the vibration-based data) are exchanged between the vibration-sensing devices 102 and the monitor 108, notwithstanding the communications connection device(s) 110 of the monitor 108 being compatible with the USB protocol and being incompatible with the NFC protocol. In such an example or another example, the signals (e.g., with the vibration-based data) are exchanged between the vibration-sensing devices 102 and the monitor 108, notwithstanding the communications connection device(s) 110 of the vibration-sensing devices 102 being compatible with the NFC protocol and being incompatible with the USB protocol.

In some examples, the communication signals include signals that encode data, which are further encrypted, or not further encrypted. The data, for instance, indicates a physiological parameter (e.g., a vibration-based physiological parameter), a treatment, an instruction, or other event detected by a transmitting device (e.g., the monitor 108, the vibration-sensing devices 102, etc.).

In various implementations, the adapter(s) 112 is communicatively connected to the monitor(s) 108 in response to the adapter(s) 112 being initialized (e.g., initialized for a communicative connection with the monitor 108). In some examples, the adapter(s) 112 is initialized by operations (or “actions”) (e.g., initialization operations or “initializations”) being performed in response to the monitor 108 detecting a connection (e.g., communication path) with the adapter(s) 112. In various cases, the monitor 108 detecting the connection between the adapter(s) 112 and the monitor 108 includes the monitor 108 performing wired device detecting operations (or “wired detecting”). In those examples or other examples, detecting the connection between the adapter(s) 112 and the monitor 108 includes detecting an electrical connection between the adapter(s) 112 and the monitor 108.

In various cases, detecting the electrical connection between the adapter(s) 112 and the monitor 108 includes the adapter(s) 112 being physically connected to (e.g., plugged into) the monitor 108 (e.g., the port(s) of the monitor 108). In those or other instances, detecting the electrical connection between the adapter(s) 112 and the monitor 108 includes the adapter(s) 112 being electrically connected to the monitor 108 (e.g., the port(s) of the monitor 108). For example, detecting the electrical connection between the adapter(s) 112 and the monitor 108 is performed in response to the adapter(s) 112 being physically connected to the monitor 108 (e.g., the port(s) of the monitor 108).

In some examples, the monitor 108 supplies power to the adapter(s) 112 in response to the adapter(s) 112 being electrically connected to the monitor 108. In those or other examples, the monitor 108 exchanges communications with the adapter(s) 112 in response to the adapter(s) 112 being electrically connected to the monitor 108. For instance, with examples in which the adapter(s) 112 receives power from, and exchanges communications with (e.g., exchanges communications via signals utilized to transfer power, data, or any combination thereof), the monitor 108, the power is received via power pins, such as a power pin of the adapter(s) 112. In such an instance or another instance, the data is exchanged by data pins, such as a data pin of the adapter(s) 112 that is recessed with respect to the power pin to enable power to be received by the adapter(s) 112 prior to a data connecting being established between the monitor 108 and the adapter(s) 112 (e.g., the data pin is recessed with respect to the power pin to enable power to be received by the adapter(s) 112 prior to data being exchanged between the adapter(s) 112 and the monitor 108).

In some cases, establishing the communicative connection between the monitor 108 and the adapter(s) 112 includes the monitor 108 identifying electrical characteristics of the adapter(s) 112, as identified electrical characteristics. For example, an electrical characteristic of the adapter(s) 112 is detected by the monitor 108 and utilized by the monitor 108 to identify the adapter(s) 112 as being electrically connected to the monitor 108. In some instances, the electrical characteristics include electrical potentials (e.g., voltage values) of data lines, such as a potential of a data line connected to, and utilized by, the monitor 108 to identify the adapter(s) 112 as being electrically connected. For instance, the data line being “pulled to a level” (e.g., “pulled to a high level”) is utilized by the monitor 108 to identify the adapter(s) 112 as being electrically connected to the monitor 108.

In some examples, communication signals are exchanged between the monitor(s) 108 and the adapter(s) 112 in response to the adapter(s) 112 being connected to the monitor 108. In some implementations, the communication signals (e.g., the adapter signals) are exchanged between the monitor 108 and the adapter(s) 112 via wired/wireless conversion. In various cases, the wired/wireless conversion includes wired signal and wireless signal conversion (e.g., USB signal and NFC signal conversion), wired protocol and wireless protocol conversion (e.g., USB protocol and NFC protocol conversion), or any combination thereof.

In some cases, establishing the communicative connection (e.g., establishing the physical connection and the electrical connection) between the monitor 108 and the adapter(s) 112 is utilized by the monitor 108 and the adapter(s) 112 to exchange the communication signals, such as signals (e.g., associated with the connection between the monitor 108 and the adapter(s) 112. In those or other examples, the signals include adapter data.

In various examples, the signals include various types of signals transmitted by the monitor 108. For example, monitor signals (e.g., signals transmitted by the monitor 108) include the signals that are in the adapter signals and that are transmitted by the monitor 108. In some cases, the signals being transmitted by the monitor 108 include request signals.

For instance, the request signals are utilized by the monitor 108 to request description data (e.g., description data, such as device descriptors) from the adapter(s) 112. For example, the request signals are utilized by the monitor 108 to query the adapter(s) 112 for the description data (e.g., the description data, which is associated with, and provided by, the adapter(s) 112), device structure data (e.g., structure data associated with, and provided by, the adapter(s) 112), any other types of data associated with the adapter(s) 112, or any combination thereof.

In various implementations, the signals include various types of signals transmitted by the adapter(s) 112. In some cases, the signals being transmitted by the adapter(s) 112 include adapter capabilities data signals (or “adapter capabilities signals”).

For instance, the adapter capabilities signals are associated with adapter capabilities. In some cases, the adapter capabilities signals are utilized by the monitor 108 to identify the adapter capabilities data. In those and other examples, the adapter capabilities signals are provided by the adapter(s) 112 and to the monitor 108.

In some cases, adapter data includes adapter capabilities data (or “capabilities data”) in the adapter capabilities signals. For example, the adapter capabilities data is associated with capabilities of the adapter(s) 112 with respect to the wired protocol (e.g., communication via the wired protocol, such as the USB protocol), the wireless protocol (e.g., communication via the wireless protocol, such as the NFC protocol), or any combination thereof. In various cases, the adapter capabilities data includes wired/wireless communicating assisting capabilities data (e.g., USB/NFC communicating assisting capabilities data).

In various cases, adapter data include adapter compatibility data (or “compatibility data”). In some examples, the adapter compatibility data is managed (e.g., identified, determined, received, etc.) via the various types of signals. In those or other examples, the compatibility data includes wired protocol compatibility data (e.g., USB compatibility data) indicating compatibility of the adapter(s) 112 with the wired protocol (e.g., the USB protocol). In those or other examples, the compatibility data includes wireless protocol compatibility data (or “compatibility data”) (e.g., NFC compatibility data) indicating compatibility of the adapter(s) 112 with the wireless protocol (e.g., short-range wireless communication protocol) (e.g., the NFC protocol). In various cases, the adapter compatibility data includes wired/wireless communicating assisting compatibility data (e.g., USB/NFC communicating assisting compatibility data).

In some examples, the various types of signals include configuration signals, initialization signals, setup signals, any other types of signals associated with connections between the monitor 108 and/or the adapter(s) 112, or any combination thereof. In those or other examples, the configuration signals are associated with configurations of connections between the monitor and/or the adapter. In those or other examples, the initialization signals are associated with initializations of connections for the monitor and/or the adapter. In those or other examples, the setup signals are associated with the setup operations for connections between the monitor and/or the adapter.

In some examples, the various types of data delivered by the signals include data utilized for operations (e.g., the adapter configurations, the adapter initializations, the adapter initializations, etc.) associated with the connections between the monitor and/or the adapter. For instance, the data delivered by the signals includes configuration data in the configuration signals, initialization data in the initialization signals, setup data in the setup signals, any other types of data in the other corresponding monitor and/or adapter signals, or any combination thereof.

In some cases, the signals associated with the connections between the monitor 108 and/or the adapter(s) 112 are associated with (e.g., are exchanged in response to, or are exchanged to initiate performance of) the operations. For instance, the operations are performed by the monitor 108, the adapter(s) 112, or any combination thereof. In various cases, the operations are utilized for establishing communications between the monitor 108 and the adapter(s) 112.

In some examples, the operations include various types of operations (or “monitor operations”) performed by the monitor 108. In those or other examples, the monitor operations (e.g., operations performed by the monitor 108) include the operations associated with the connections between the monitor 108 and/or the adapter(s) 112. In those or other examples, wired/wireless operations (e.g., operations performed utilizing the wired protocol and the wireless protocol) include the monitor operations.

In those or other examples, the operations performed by the monitor 108 include configuration operations (or “configurations”) (e.g., configuration operations associated with configuration of the adapter(s) 112), initialization operations (or “initializations”) (e.g., initialization operations associated with initialization of the adapter(s) 112), setup operations (or “setups”) (e.g., setup operations associated with setup of the adapter(s) 112), any other monitor and/or adapter operations, or any combination thereof.

In various implementations, the operations associated with the connections between the monitor 108 and/or the adapter(s) 112 include various types of adapter assisting operations (or “assisting operations”)) performed by the adapter(s) 112. In those or other examples, the assisting operations include configuration assisting operations, initialization assisting operations, setup assisting operations, any other assisting operations, or any combination thereof.

In some cases, the operations (e.g., the assisting operations, etc.) performed by the adapter(s) 112 enable the adapter(s) 112 to be identified by, configured, initialized, setup, and communicatively connected to, the monitor 108. In those or other examples, the adapter data (e.g., the capabilities data, the compatibility data, the configuration data, the initialization data, the setup data, any other types of data in corresponding monitor and/or adapter signals, or any combination thereof) is utilized for performance of the operations performed by the monitor 108 and/or the adapter(s) 112.

In various cases, the monitor 108 utilizes adapter drivers to identify the adapter(s) 112 as being compatible with the wireless protocol (e.g., the short-range wireless communication protocol) (e.g., the NFC protocol). For example, the monitor 108 identifies adapter drivers in response to the signals from the adapter(s) 112, the adapter data, or any combination thereof.

In some implementations, the adapter drivers are utilized to establish the communication connection between the monitor 108 and the adapter(s) 112. In various cases, the monitor 108 utilizes adapter drivers operations to identify the adapter drivers. For instance, identifying the adapter drivers includes loading the driver data, storing the driver data (e.g., storing the driver data in the storage device of the monitor 108), installing the driver data, performing any other operations (e.g., driver operations associated with the various types of signals, the adapter data, or any combination thereof), or any combination thereof.

In various cases, the signals utilized by the monitor 108 to manage (e.g., to perform various operations associated with the adapter drivers) the adapter are utilized to manage the adapter drivers in various ways. For instance, with examples in which the signals are received from the adapter(s) 112 and by the monitor 108, the monitor 108 performs adapter drivers operations. In some cases, the monitor operations include operations that are included in the adapter drivers operations and that are performed by the monitor 108.

In some examples, the adapter driver operations include identifying adapter drivers, loading the adapter drivers, storing the adapter drivers (e.g., storing the adapter drivers in the storage device of the monitor 108), installing the adapter driver, performing any other operations associated with the adapter drivers, or any combination thereof, in response to the signals received from the adapter(s) 112. In those or other examples, the monitor 108 performs any of the adapter driver operations utilizing the adapter capabilities signals, the signals received from the adapter(s) 112 and/or from the vibration-sensing devices 102 (e.g., via the adapter(s) 112), or any combination thereof.

In various implementations, the adapter drivers are utilized by the monitor 108 to enable, activated, control, etc., the adapter(s) 112 to participate and/or assist in operations (e.g., the monitor operations) performed by the monitor 108. In those or other examples, adapter operations, which are utilized by the adapter(s) 112 to participate and/or assist in the monitor operations, include wired/wireless assisting operations. For instance, the wired/wireless operations include the wired/wireless assisting operations.

In some cases, the wired/wireless operations performed by the monitor 108 include conversion operations, detection operations, pairing operations, communicating operations, any other wired/wireless operations of various types, or any combination thereof. In some cases, the wired/wireless assisting operations performed by the adapter(s) 112 include conversion assisting operations, detection assisting operations, pairing assisting operations, communicating assisting operations, any other wired/wireless assisting operations of various types, or any combination thereof.

While any of the monitor operations, any of the wired/wireless operations, or any combination thereof, are performed by the monitor 108, as discussed above in the current disclosure, it is not limited as such. In some examples, the monitor operations, any of the wired/wireless operations, or any combination thereof, are performed by the monitor 108, the adapter(s) 112, or any combination thereof.

In some implementations, the monitor 108 and the adapter(s) 112 are enabled, activated, controlled, etc., in response to the adapter drivers. In some examples, the monitor 108 and the adapter(s) 112 are enabled, activated, controlled, etc., to perform the wired/wireless converting operations and the wired/wireless converting assisting operations, respectively. In those or other examples, the monitor 108 and the adapter(s) 112 are enabled, activated, controlled, etc., to perform the wired/wireless detecting operations and the wired/wireless detecting assisting operations, respectively. In those or other examples, the monitor 108 and the adapter(s) 112 are enabled, activated, controlled, etc., to perform the wired/wireless pairing operations and the wired/wireless pairing assisting operations, respectively. In those or other examples, the monitor 108 and the adapter(s) 112 are enabled, activated, controlled, etc., to perform the wired/wireless communicating operations and the wired/wireless communicating assisting operations, respectively. In those or other examples, the monitor 108 and the adapter(s) 112 are enabled, controlled, activated, etc., to perform any other types of wired/wireless operations, including wired/wireless monitor operations and wired/wireless adapter operations, respectively, in response to the adapter drivers.

In some examples, the adapter drivers are utilized by the monitor 108 to enable, activate, control, etc., the adapter(s) 112 to assist in the wired/wireless converting operations (or “converting operations”) (or “wired/wireless converting”) (e.g., USB/NFC converting operations (or “USB/NFC converting”). In those or other examples, the adapter drivers include a device driver that is utilized by the monitor 108 to enable the adapter(s) 112 to assist in the converting operations.

In some examples, the adapter drivers are utilized by the monitor 108 to enable, activate, control, etc., the adapter(s) 112 to assist in the wired/wireless detecting operations (or “detecting operations”) (or “wired/wireless detecting”) (e.g., detecting, which includes the wired/wireless converting) (e.g., USB/NFC detecting operations (or “USB/NFC detecting”). In those or other examples, the adapter drivers include a device driver that is utilized by the monitor 108 to enable the adapter(s) 112 to assist in the detecting operations.

In various examples, the adapter drivers are utilized by the monitor 108 to enable, activate, control, etc., the adapter(s) 112 to assist in the wired/wireless pairing operations (or “pairing operations”) (or “pairing”) (e.g., pairing, which includes the wired/wireless converting) (e.g., USB/NFC pairing operations (or “USB/NFC pairing”). In some examples, the adapter drivers include a device driver (or “driver”) that is utilized by the monitor 108 to control, enable, etc., the adapter(s) 112 to assist in pairing operations. In those or other examples, the adapter drivers include a device driver that is utilized by the monitor 108 to enable the adapter(s) 112 to assist in the pairing operations.

In various examples, the adapter drivers are utilized by the monitor 108 to enable, activate, control, etc., the adapter(s) 112 to assist in the wired/wireless communicating operations (or “communicating operations”) (or “communicating”) (e.g., communicating, which includes the wired/wireless converting) (e.g., USB/NFC communicating operations (or “USB/NFC communicating”). In those or other examples, the adapter drivers include a device driver that is utilized by the monitor 108 to enable the adapter(s) 112 to assist in wireless device the communicating operations.

While the adapter drivers utilized to enable, activate, control, etc., the monitor 108 and the adapter(s) 112 are separate from one another, as discussed above in the current disclosure, it is not limited as such. In some examples, any of the adapter drivers are combined with (e.g., integrated together with) any of the other adapter drivers, for purposes of enabling, activating, controlling, etc., the monitor 108 and the adapter(s) 112 for performing any of the corresponding functions.

In some implementations, activation, which includes activation (or “adapter activation”) of the adapter(s) 112, is performed. In various examples, the activation includes activating operations being performed by the monitor 108, the adapter(s) 112, or any combination thereof, to activate the adapter(s) 112 for adapter operations. In various examples, the activating operations are performed in response to the connection (e.g., the physical connection and the electrical connection between the monitor 108 and the adapter(s) 112), the identified electrical characteristics, the signals (e.g., associated with the connection between the monitor 108 and the adapter(s) 112), the adapter data, the adapter drivers, the monitor and/or adapter operations etc., or any combination thereof.

In some cases, the activating operations include monitor activating operations and adapter activating operations. In those or other examples, the monitor activating operations include operations (e.g., operations in the monitor operations utilized for activation) that are performed to activate the monitor 108, such as to perform the monitor operations associated with monitor activation. In those or other examples, the adapter activating operations include operations (e.g., operations in the adapter operations utilized for activation) that are performed to activate the adapter(s) 112, such as to perform the adapter operations associated with adapter activation.

In various cases, the adapter(s) 112 is enabled, activated, etc., to perform individual ones of the assisting operations in response to successful completion of activation of the corresponding assisting operations. In some examples, the monitor activation includes wired/wireless converting operations activation, wired/wireless detecting operations activation, wired/wireless pairing operations activation, wired/wireless communicating operations activation, any other adapter activation operations (e.g., activation of any other any other assisting operations), or any combination thereof. In those or other examples, the adapter activation includes wired/wireless converting assisting operations activation, wired/wireless detecting assisting operations activation, wired/wireless pairing assisting operations activation, wired/wireless communicating assisting operations activation, any other adapter activation operations (e.g., activation of any other any other assisting operations), or any combination thereof.

In some examples, the monitor 108 is enabled, activated, etc., to perform wired/wireless converting operations in response to completion of the wired/wireless converting operations activation (e.g., activation of wired/wireless converting operations) (e.g., successful completion of wired/wireless converting activation operations). In those or other examples, the adapter(s) 112 is enabled, activated, etc., to perform the wired/wireless detecting operations in response to completion of the wired/wireless detecting operations activation (e.g., activation of wired/wireless detecting operations) (e.g., successful completion of wired/wireless detecting activation operations). In those or other examples, the adapter(s) 112 is enabled, activated, etc., to perform the wired/wireless pairing operations in response to completion of the wired/wireless pairing operations activation (e.g., activation of the wired/wireless pairing operations) (e.g., successful completion of wired/wireless pairing activation operations). In those or other examples, the adapter(s) 112 is enabled, activated, etc., to perform wired/wireless communicating operations in response to completion of wired/wireless communicating operations activation (e.g., activation of the wired/wireless communicating operations) (e.g., successful completion of wired/wireless communicating activation operations).

In some examples, the activation of any the wired/wireless operations is utilized to enable the monitor 108 to perform the corresponding wired/wireless operations. In those or other examples, the activation of the wired/wireless converting operations is utilized to enable the monitor 108 to perform the wired/wireless converting operations. In those or other examples, the activation of the wired/wireless detecting operations is utilized to enable the monitor 108 to perform the wired/wireless detecting operations. In those or other examples, the activation of the wired/wireless pairing operations is utilized to enable the monitor 108 to perform the wired/wireless pairing operations. In those or other examples, the activation of the wired/wireless communicating operations is utilized to enable the monitor 108 to perform the wired/wireless converting operations. In those or other examples, the activation of the wired/wireless communicating operations is utilized to enable the monitor 108 to perform the wired/wireless converting operations. In those or other examples, the activation of any others of the wired/wireless operations is utilized to enable the monitor 108 to perform any others of the wired/wireless operations.

In some examples, the adapter(s) 112 is enabled, activated, etc., to perform the wired/wireless converting assisting operations in response to completion of the wired/wireless converting assisting operations activation (e.g., activation of wired/wireless converting assisting operations) (e.g., successful completion of wired/wireless converting assisting activation operations). In those or other examples, the adapter(s) 112 is enabled, activated, etc., to perform the wired/wireless detecting assisting operations in response to completion of the wired/wireless detecting assisting operations activation (e.g., activation of wired/wireless detecting assisting operations) (e.g., successful completion of wired/wireless detecting assisting activation operations). In those or other examples, the adapter(s) 112 is enabled, activated, etc., to perform the wired/wireless pairing assisting operations in response to completion of the wired/wireless pairing assisting operations activation (e.g., activation of the wired/wireless pairing assisting operations) (e.g., successful completion of wired/wireless pairing assisting activation operations). In those or other examples, the adapter(s) 112 is enabled, activated, etc., to perform wired/wireless communicating assisting operations in response to completion of wired/wireless communicating assisting operations activation (e.g., activation of the wired/wireless communicating assisting operations) (e.g., successful completion of wired/wireless communicating assisting activation operations).

In some examples, the activation of any the wired/wireless assisting operations is utilized to enable the adapter(s) 112 to assist in the corresponding wired/wireless operations being performed by the monitor 108. In those or other examples, the activation of the wired/wireless converting assisting operations is utilized to enable the adapter(s) 112 to assist in the wired/wireless converting operations being performed by the monitor 108. In those or other examples, the activation of the wired/wireless detecting assisting operations is utilized to enable the adapter(s) 112 to assist in the wired/wireless detecting operations being performed by the monitor 108. In those or other examples, the activation of the wired/wireless pairing assisting operations is utilized to enable the adapter(s) 112 to assist in the wired/wireless pairing operations being performed by the monitor 108. In those or other examples, the activation of the wired/wireless communicating assisting operations is utilized to enable the adapter(s) 112 to assist in the wired/wireless converting operations being performed by the monitor 108. In those or other examples, the activation of the wired/wireless communicating assisting operations is utilized to enable the adapter(s) 112 to assist in the wired/wireless converting operations being performed by the monitor 108. In those or other examples, the activation of any others of the wired/wireless assisting operations is utilized to enable the adapter(s) 112 to assist in any others of the wired/wireless operations being performed by the monitor 108.

In some examples, the monitor 108 identifies the adapter(s) 112 as being compatible with the wired protocol (e.g., the USB protocol), the wireless protocol (e.g., the NFC protocol), or any combination thereof. For instance, the monitor 108 identifies the adapter(s) 112 as being compatible with the wired protocol, in response to the connection (e.g., the physical connection and the electrical connection between the monitor 108 and the adapter(s) 112), the electrical characteristics, the signals (e.g., associated with the connection between the monitor 108 and the adapter(s) 112), the adapter data, the adapter drivers, the monitor and/or adapter operations, the activation operations, etc., or any combination thereof.

In some implementations, the monitor 108 and the adapter(s) 112 perform various operations utilizing the connection (e.g., the physical connection and the electrical connection between the monitor 108 and the adapter(s) 112), the electrical characteristics, the various types of signals (e.g., with the adapter data), the adapter drivers, the monitor and/or adapter operations, the activation operations, etc., or any combination thereof. For example, the monitor 108 and the adapter(s) 112 perform the wired/wireless converting operations and the wired/wireless converting assisting operations, respectively, the wired/wireless detecting operations and the wired/wireless detecting assisting operations, respectively, the wired/wireless pairing operations and the wired/wireless pairing assisting operations, respectively, the wired/wireless communicating operations and the wired/wireless communicating assisting operations, respectively, any of the other wired/wireless operations and any of the other wired/wireless assisting operations, respectively, utilizing the connection (e.g., the physical connection and the electrical connection between the monitor 108 and the adapter(s) 112), the electrical characteristics, the signals (e.g., associated with the connection between the monitor 108 and the adapter(s) 112), the adapter data, the adapter drivers, the monitor and/or adapter operations, the activation operations, etc., or any combination thereof.

In some cases, any of various operations (e.g., the monitor and/or adapter operations) (e.g., wired/wireless operations, such as the monitor operations, the adapter operations, any other wired/wireless operations, or any combination thereof), are performed in response to the adapter(s) 112 satisfying various characteristics (e.g., NFC characteristics). For example, any of the various operations (e.g., the monitor and/or adapter operations) (e.g., wired/wireless operations, such as the monitor operations, the adapter operations, any other wired/wireless operations, or any combination thereof) are performed in response to the adapter(s) 112 satisfying a power characteristic (e.g., a short-range wireless communication power characteristic), a wavelength characteristic (e.g., a short-range wireless communication wavelength characteristic), a frequency characteristic (e.g., a short-range wireless communication frequency characteristic), or any combination thereof, associated with the wireless protocol. In some cases, the monitor operations are performed utilizing the antenna of the adapter(s) 112, the antenna including a short-range wireless communication antenna satisfying the short-range wireless communication power characteristic, the short-range wireless communication wavelength characteristic, the short-range wireless communication frequency characteristic, any other wireless protocol characteristic, or any combination thereof, associated with the wireless protocol.

In some instances, any of the various operations (e.g., the monitor and/or adapter operations) (e.g., wired/wireless operations, such as the monitor operations, the adapter operations, any other wired/wireless operations, or any combination thereof) are performed in response to the adapter(s) 112 satisfying a short-range wireless communication power characteristic (e.g., an NFC power characteristic, such as a power with a value of 6 milliwatts (e.g., 1 milliwatt, 5 milliwatts, 10 milliwatts, etc., with a variation of 100 microwatts, 1 milliwatt, 5 milliwatts, etc.) and a voltage with a value of 3 volts (e.g., 3 volts with a variation of 0.1 volt, 0.5 volt, etc.)), the short-range wireless communication wavelength characteristic (e.g., an NFC wavelength characteristic, such as a wavelength with a value of 22 meters, with a variation of 1 meter, 2 meters, etc.), the short-range wireless communication frequency characteristic (e.g., an NFC frequency characteristic, such as a frequency with a value in a frequency band (e.g., a frequency with a value in a 13.56 megahertz unlicensed radio frequency industrial, scientific and medical (ISM) band, with a variation of 10 kilohertz, 1 megahertz, etc.)). For example, any of the various operations (e.g., the monitor and/or adapter operations) (e.g., wired/wireless operations, such as the monitor operations, the adapter operations, any other wired/wireless operations, or any combination thereof) are performed in response to the antenna of the adapter(s) 112 including the short-range wireless communication power characteristic, the short-range wireless communication wavelength characteristic, the short-range wireless communication frequency characteristic, any other wireless protocol characteristic, or any combination thereof, associated with the wireless protocol.

In various implementations, wireless fields (e.g., a wireless field associated with the monitor 108, the communications connection device(s) 110, and/or the adapter(s) 112, a wireless field associated with any of the vibration-sensing devices 102, the communications connection device(s) 110, or any combination thereof) are generated in response to the connection (e.g., the physical connection and the electrical connection between the monitor 108 and the adapter(s) 112), the identified electrical characteristics, the signals (e.g., associated with the connection between the monitor 108 and the adapter(s) 112), the adapter data, the adapter drivers, the monitor and/or adapter operations, the activation operations, etc., or any combination thereof. In some cases, any of the various operations (e.g., wired/wireless operations, such as the monitor operations, the adapter operations, any other wired/wireless operations, or any combination thereof) are performed utilizing (e.g., in response to) the generation of the wireless fields. In some cases, the monitor 108 is utilized (e.g., utilized as an “initiator device”), in combination with the adapter(s) 112, to produce (e.g., generate) a wireless field (e.g., an RF field) (e.g., an NFC field).

For instance, with the monitor 108 (e.g., the electrical device, the communications connection device(s) 110, the adapter(s) 112, or any combination thereof) being utilized to generate a wireless field (e.g., an NFC field) via field generation operations included in the wired/wireless operations, the monitor 108 utilizes (e.g., controls) the adapter(s) 112 (e.g., the monitor 108 controls the transceiver (e.g., the antenna) in the adapter(s) 112) to produce radio signals (e.g., RF signals compatible with the wireless protocol) (e.g., RF signals compatible with the NFC protocol) (e.g., NFC signals).

In some examples, generating the wireless field includes the monitor 108 utilizing the adapter(s) 112 to transmit the radio signals, as transmitted signals (e.g., transmitted wireless signals) (e.g., transmitted NFC signals). In those or other examples, the generating of the wireless field includes the monitor 108 utilizing the adapter(s) 112 to generate the wireless field (e.g., the NFC field) via the transmitted signals. In those or other examples, wireless fields (e.g., wireless fields including one or more of the wireless field), which are generated by the monitor 108 and the adapter(s) 112, create an electromagnetic wave (e.g., electromagnetic radiation). The wireless fields (e.g., the electromagnetic wave/radiation), for example, form a wireless signal (e.g., a radio signal, such as an RF signal) (e.g., an NFC signal).

In some implementations, the generating of the wireless field includes the monitor 108 transmitting a control signal (e.g., a control signal compatible with the wired protocol) (or “wired control signal”) (e.g., a control signal compatible with the USB protocol) (or “USB control signal”) to the adapter(s) 112, causing the adapter(s) 112 to generate the wireless field (e.g., (e.g., the wired control signal causes the adapter(s) 112 to assist in generation of the wireless field).

In some examples, the adapter(s) 112 is caused to generate the wireless field (e.g., the wireless field is formed via the wireless signal being emitted by the adapter(s) 112, such as by the transceiver (e.g., the antenna) of the adapter(s) 112) in response to the wired control signal (e.g., the USB control signal). In those or other examples, the wireless field is generated in response to power supplied by the monitor 108 and to the adapter(s) 112. In some instances, the wireless field, such as the NFC field, is generated by the monitor 108 being operated in combination with the adapter(s) 112, as a combined monitor.

In some implementations, the wireless field is generated in response to the wired control signal, the power (e.g., alternating current (AC) power) (e.g., an AC power signal) (or “power signal”) received from the monitor 108, or any combination thereof. In various examples, the wired control signal, such as the USB control signal causes the adapter(s) 112 to utilize the AC power to generate the wireless field (e.g., the wired control signal is utilized to instruct and control the adapter(s) 112 to generate the wireless field, for instance, with examples in which the control signal is separate from the power signal).

In various cases, the AC signal that is transmitted by the monitor 108 and to the adapter(s) 112 is utilized, as the wired control signal, to control the adapter(s) 112 to generate the wireless field (e.g., the AC signal is utilized as both the power signal and the control signal, as a combined signal) (e.g., the wired control signal is provided via the AC signal). In some examples, the wired control signal, the power signal, or any combination thereof, are communicated from the monitor 108 and to the adapter(s) 112, and utilized to generate the wireless field, via the wired/wireless converting (e.g., wired/wireless power signal converting).

In some implementations, various devices (e.g., the monitor 108, the communications connection device(s) 110, the adapter(s) 112, the vibration-sensing devices 102, the communications connection device(s) 110, or any combination thereof) perform the wired/wireless operations, such as the converting, the detecting, the pairing, the communicating, any other wired/wireless operations, or any combination thereof. In various examples, the wired/wireless operations are performed via inductive coupling between the adapter(s) 112 and the other wireless device (e.g., any of the vibration-sensing devices 102 (e.g., the communications connection device(s) 110)).

For instance, with examples in which the wireless device detecting includes the inductive coupling between the adapter(s) 112 and any of the vibration-sensing devices 102, transceivers (e.g., two transceivers (e.g., a transceiver in the adapter(s) 112 and a transceiver in the communications connection device(s) 110) with two corresponding antennas (e.g., two nearby loop antennas)), form an air-core transformer (or “antenna coil”). In some examples, the air-core transformer is formed in response to any of the vibration-sensing devices 102 (e.g., the communications connection device(s) 110) being moved toward the adapter(s) 112, held in front of the adapter(s) 112, moved or held in any other way with respect to the adapter(s) 112, or any combination thereof. In those or examples, the air-core transformer is formed in response to relative movement, or lack thereof, between the adapter(s) 112 (e.g., the transceiver in the adapter(s) 112) and the communications connection device(s) 110 (e.g., the transceiver in communications connection device(s) 110), in response to the wire/wireless communications (e.g., the wired communications that are transmitted by the monitor 108 and converted utilizing the adapter(s) 112).

In those or other examples, any of the wired/wireless operations are performed (e.g., the wired/wireless operations being performed between the vibration-sensing device(s) 102 and the monitor 108) (e.g., formation of the air-core transformer is initiated) (e.g., formation of the air core transformer is performed) (e.g., the air-core transformer is formed) in response to a distance between any of the vibration-sensing devices 102 and the monitor 108 being less than a threshold distance (e.g., a maximum distance) (e.g., 4 centimeters, 10 centimeters, 20 centimeters, etc., with a variation of 1 millimeters, 1 centimeter, etc.). In various cases, any of the vibration-sensing devices 102 being in a “proximity” of the monitor 108 includes any of the vibration-sensing devices 102 and the monitor 108 being positioned with the distance between any of the vibration-sensing devices 102 and the monitor 108 being less than the threshold distance.

In those or other examples, the monitor 108 transmitting the wired control signal to the adapter(s) 112 to generate the wireless field (e.g., the NFC field) enables the air-core transformer to be formed, in response to the distance between the monitor 108 (e.g., the adapter(s) 112) and any of the vibration-sensing devices 102 (e.g., the communications connection device(s) 110) being less than the threshold distance.

In various cases, transmitting of a field generating signal (e.g., the wired control signal) includes transmitting (e.g., transmitted via the wired/wireless converting) of a pulse signal (or “pulse”) or a continuous signal. In some examples, for instance with the wired control signal including the pulse, the adapter(s) 112 is utilized by the monitor 108 to maintain generation of the wireless field at an ongoing basis (e.g., until another pulse signals, such as a “stop,” signal, is transmitted by the monitor 108, (e.g., transmitted via the wired/wireless converting). In another example, for instance with the wired control signal including the continuous signal, the adapter(s) 112 is utilized by the monitor 108 to maintain generation of the wireless field at an ongoing basis (e.g., until the transmitting of the continuous signal stops).

In some instances, for example with the wireless field being an NFC field, such as an alternating magnetic field being utilized as a “primary coupling factor” between the monitor 108 (e., the adapter(s) 112) and any of the vibration-sensing devices 102 (e.g., the communications connection device(s) 110), power being radiated (e.g., radiated from the wireless field) (e.g., radiated, externally) in a form of radio waves (e.g., electromagnetic waves, also involving an oscillating electric field), is maintained below a threshold power. By maintaining the power below the threshold power, for instance with examples in which the wireless field includes the NFC field being generated in response to the wired control signal, such as the USB control signal, a level of interference between the devices (e.g., the monitor 108, any of the vibration-sensing devices 102, or any combination thereof) and other devices (e.g., other NFC devices, other types of devises, or any combination thereof), such as a level of interference associated with any radio communications at a same frequency as, or different frequency from, the AC power signal (e.g., the different frequency including another frequency that has a value that is greater than, or less than, a frequency of the AC signal transmitted by the monitor 108, with a difference between the value of the other frequency and a value of a frequency of the AC signal transmitted by the monitor 108 being less than a threshold difference), is maintained below a threshold interference level. Because an importance of providing reliable treatment to subjects is relatively high in certain scenarios, such as in emergency scenarios, utilizing USB/NFC operations enable electronical interference between electronic devices to be minimized (e.g., such as for instances in which the monitor 108, any of the vibration-sensing devices 102, or any combination thereof, are medical devices), thereby increasing reliability of treatment provided via the electronic devices.

In some cases, any of the vibration-sensing devices 102 do not include a power supply. For instance, with examples in which a vibration-sensing device 102 does not include the power supply, the vibration-sensing device 102 without the power supply receives power from the wireless field generated by the vibration-sensing device 102 (e.g., any of the vibration-sensing devices 102 utilizes the communications connection device(s) 110 to receive power, from the monitor 108, such as from the communications connection device(s) 110, via the wired/wireless operations enabled via the adapter(s) 112).

In those or other examples, such as with the vibration-sensing device 102 not including the power supply, the vibration-sensing device 102 receives power from a magnetic field (e.g., a magnetic field as part of the wireless field) generated by the vibration-sensing device 102. For instance, with examples in which the vibration-sensing device 102 does not include the power supply, the vibration-sensing device 102 communicates with the monitor 108 (e.g., the vibration-sensing devices 102 and the monitor 108 utilize the communications connection devices 104 and 110 respectively, to communicate, via the wired/wireless operations enabled via the adapter(s) 112) via a first mode of wireless communication included in various wireless communication modes (e.g., various modes utilized to exchange wireless communications, transmit/receive wireless signals, or any combination thereof, using the wireless protocols).

In various cases, such as for the examples in which the vibration-sensing device 102 without the power supply utilizes the first mode of wireless communication, the wired/wireless data is exchanged between the vibration-sensing device 102 and the monitor 108 by the vibration-sensing device 102 utilizing (e.g., modulating) the wireless field (e.g., the wired control signal initiated wireless field) (e.g., the USB control signal initiated NFC field) for communications (e.g., the vibration-sensing device 102 “acts” as a “transponder,” such as by receiving signals, and, upon receiving, signals, emits different signals in response). In those cases, any wired/wireless operations (e.g., the USB/NFC operations), such as the converting, the detecting, the pairing, the communicating, any other wired/wireless operations, or any combination thereof, are performed utilizing the wireless field for communications (e.g., the wired/wireless operations are performed via wireless field modulating, which is utilized by the monitor 108, the communications connection device(s) 110, the adapter(s) 112, the vibration-sensing device 102, the communications connection device(s) 110, to generate wired/wireless data, which is exchanged by communications with the monitor 108 via the wired/wireless converting).

Alternatively, any of the vibration-sensing devices 102 includes a power supply. For instance, with examples in which a vibration-sensing device 102 includes the power supply, the vibration-sensing device 102 and the monitor 108 communicate (e.g., the vibration-sensing devices 102 and the monitor 108 utilize the communications connection devices 104 and 110 respectively, to communicate, via the wired/wireless operations enabled via the adapter(s) 112) via a second mode of wireless communication from among various modes of wireless communications.

In various cases, such as for the examples in which the vibration-sensing device 102 including the power supply utilizes the second mode of wireless communication, the vibration-sensing device 102 and the monitor 108 communicate to exchange the wired/wireless data by alternately generating their own fields (or “alternatively generated fields”) (e.g., wireless fields (e.g., NFC fields). In those cases, for instance, a device (e.g., the monitor 108 or the vibration-sensing device 102) transmits data, and the other device stops transmitting in order to receive the transmitted data. In those examples, any wired/wireless operations (e.g., the USB/NFC operations), such as the converting, the detecting, the pairing, the communicating, any other wired/wireless operations, or any combination thereof, are performed utilizing the alternatively generated fields (e.g., the wired/wireless operations are performed via the alternatively generated fields, which are utilized by the monitor 108, the communications connection device(s) 110, the adapter(s) 112, the vibration-sensing device 102, the communications connection device(s) 110, to generate wired/wireless data, which is exchanged by communications with the monitor 108 via the wired/wireless converting).

In various embodiments, any of the vibration-sensing devices 102 and the monitor 108 perform the wired/wireless operations (e.g., the vibration-sensing devices 102 and the monitor 108 utilize the communications connection devices 104 and 110 respectively, to communicate, via the wired/wireless operations enabled via the adapter(s) 112) via the first mode of wireless communication, the second mode of wireless communication, any other wireless communication modes from among the various modes of wireless communications, or any combination thereof. For example, any of the vibration-sensing devices 102 and the monitor 108 exchange the wired/wireless communications via any types of NFC protocol communication modes, any other types of other wireless communication modes associated with any other wireless protocol, or any combination thereof.

In some examples, the wired/wireless operations include operations that are in the wireless device converting operations and that are performed by the monitor 108, which utilizes the adapter(s) 112 (e.g., the wireless device converting assisting operations performed by the adapter(s) 112). In various implementations, the wireless device converting operations performed by the monitor 108, which utilizes the adapter(s) 112, include converting of wired signals to wireless signals, converting of wired protocols to wireless protocols, converting of wireless signals to wired signals, converting of wireless protocols to wired protocols, and any combination thereof.

In some examples, the converting includes utilizing corresponding electronic devices (e.g., electronic sub-devices) (e.g., circuits, processors, memory, etc.) of the monitor 108 (e.g., a circuit that is included in the monitor 108 and that electrically connects any portions (e.g., devices, sub-devices, etc.) of the monitor 108), the communications connection device(s) 110 (e.g., a circuit that is included in the communications connection device(s) 110 and that electrically connects any portions (e.g., devices, sub-devices, etc.) of the communications connection device(s) 110), the adapter(s) 112 (e.g., a circuit that is included in the adapter(s) 112 and that electrically connects any portions (e.g., devices, sub-devices, etc.) of the adapter(s) 112), and any combination thereof, to convert wired signals to wireless signals, convert wired protocols to wireless protocols, convert wireless signals to wired signals, convert wireless protocols to wired protocols, and any combination thereof. In those or other examples, the converting includes utilizing antennas (e.g., an antenna of the adapter(s) 112) to convert wired signals to wireless signals, convert wireless signals to wired signals, and any combination thereof.

In some examples, the converting includes utilizing the corresponding circuits of the monitor 108, the communications connection device(s) 110, the adapter(s) 112, and any combination thereof, and utilizing the antennas (e.g., an antenna of the adapter(s) 112) to convert wired signals transmitted by the monitor 108 to wireless signals transmitted by the adapter(s) 112 (e.g., and to the communications connection device(s) 110). In some examples, the converting includes utilizing the corresponding circuits of the monitor 108, the communications connection device(s) 110, the adapter(s) 112, and any combination thereof, and utilizing antennas (e.g., an antenna of the adapter(s) 112) to convert wireless signals received by adapter(s) 112 (e.g., and from the communications connection device(s) 110) to wired signals received by the monitor 108.

In some examples, the wired/wireless operations include operations that are in the wireless device converting operations and that are performed by any of the vibration-sensing devices 102, which utilizes the communications connection device(s) 110. In various implementations, the wireless device converting operations performed by any of the vibration-sensing devices 102, which utilizes the communications connection device(s) 110, include converting of wired signals to wireless signals, converting of wired protocols to wireless protocols, converting of wireless signals to wired signals, converting of wireless protocols to wired protocols, and any combination thereof.

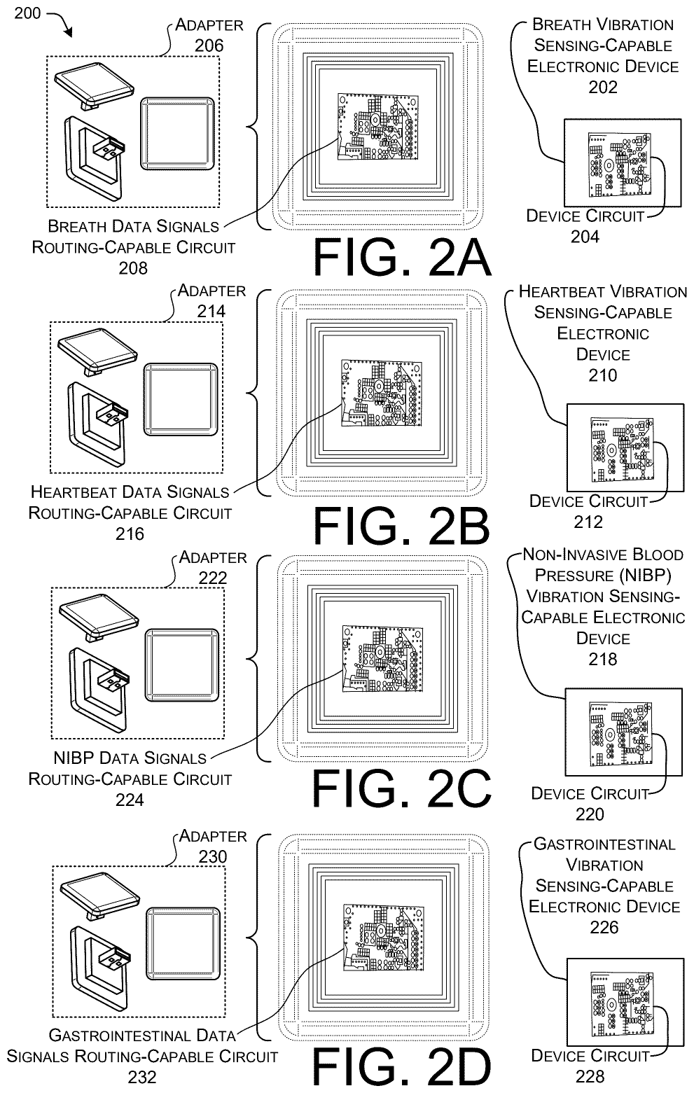

In some examples, the converting includes utilizing corresponding electronic devices (e.g., electronic sub-devices) (e.g., circuits, processors, memory, etc.) (e.g., the circuits 204, 212, 220, and/or 228, as discussed below in FIGS. 2A-2D, respectively), of the vibration-sensing devices 102 (e.g., a circuit that is included in individual ones of the vibration-sensing devices 102 and that electrically connects any portions (e.g., devices, sub-devices, etc.) of any of the vibration-sensing devices 102). In those or other examples, the converting includes utilizing the communications connection device(s) 110 (e.g., a circuit that is included in the communications connection device(s) 110 and that electrically connects any portions (e.g., devices, sub-devices, etc.) of the communications connection device(s) 110). In those or other examples, the converting includes utilizing the antennas (e.g., an antenna in the communications connection device(s) 110), to convert wireless signals received by communications connection device(s) 110 (e.g., and from the adapter(s) 112) to wired signals received by the vibration-sensing devices 102. In those or other examples, the converting includes utilizing corresponding circuits of the vibration-sensing devices 102, the communications connection device(s) 110, and any combination thereof, and utilizing the antennas (e.g., the communications connection device(s) 110), to convert wired signals transmitted the monitor 108 to wireless signals transmitted by the communications connection device(s) 110 (e.g., and to the adapter(s) 112).

In some implementations, wired/wireless conversions (e.g., USB/NFC conversions) include conversions between the wired and wireless signals (or “wired/wireless converting”) (e.g., conversions between the USB and NFC signals (or “USB/NFC converting”)), conversions between the wired and wireless protocols (e.g., USB and NFC protocols), or any combination thereof. For instance, the USB/NFC converting is performed via various operations (e.g., USB and NFC signals conversion operations, USB and NFC protocols conversion operations, or any combination thereof).

In some examples, USB/NFC conversion operations are performed by the monitor 108, the adapter(s) 112, or any combination thereof. In those or other examples, the USB/NFC conversions include USB-NFC conversions (e.g., conversions of signals in compliance with the USB protocol to signals in compliance with the NFC protocol) (e.g., conversions from the USB to NFC protocols), NFC-USB conversions (e.g., conversions of signals in compliance with the NFC protocol to signals in compliance with the USB protocol) (e.g., conversions from the NFC to USB protocols), or any combination thereof.

In some examples, for instance with the communications connection device(s) 110 including, or being included in, a USB device, USB/NFC conversion operations are performed by any of the vibration-sensing devices 102, the USB device, or any combination thereof. In those or other examples, the USB/NFC conversion operations include USB-NFC conversions (e.g., conversions of signals in compliance with the USB protocol to signals in compliance with the NFC protocol) (e.g., conversions from the USB to NFC protocols), NFC-USB conversions (e.g., conversions of signals in compliance with the NFC protocol to signals in compliance with the USB protocol) (e.g., conversions from the NFC to USB protocols), or any combination thereof.

Although, in some instances, the communications connection device(s) 110 includes, or is included in, the USB device, as discussed above in the current disclosure, it is not limited as such. In some examples, for instance with the communications connection device(s) 110 not including, or being included in, any USB device, no USB/NFC conversion is performed by a corresponding vibration-sensing device 102, the communications connection device(s) 110, and any combination thereof, and the antenna in the communications connection device(s) 110.

In some examples, the wired/wireless operations include the wireless device detecting operations being performed, by the monitor 108, which utilizes the adapter(s) 112 (e.g., the wireless device detecting assisting operations performed by the adapter(s) 112). In various implementations, the wireless device detecting operations include detecting (e.g., via the converting) of another wireless device (e.g., any of the vibration-sensing devices 102).

In some examples, the monitor 108 utilizes (e.g., identifies, determines, stores, generates, modifies data) data (e.g., stores the data in a storage device (e.g., memory, a hard drive, or any number of storage devices of any types) of the monitor 108) that includes wireless detection data (e.g., data representing a wireless detection status) (e.g., data stored in the storage device of the monitor 108) to indicate whether the wireless device (e.g., any of the vibration-sensing devices 102) is detected. In those or other examples, the wired/wireless data includes the wireless detection data being managed (e.g., identified, determined, generated, modified, etc.) in response to the wired/wireless data indicating any of the vibration-sensing devices 102 (e.g., the communications connection device(s) 110) is detected (e.g., via any of the various wired/wireless communication modes, or any combination thereof).

In those or other examples, the wireless detection data associated with a corresponding one of the vibration-sensing devices 102 being detected includes a value of “detected,” and the wireless detection data associated with a corresponding one of the vibration-sensing devices 102 not being detected includes a value of “undetected” (e.g., an initial value of the wireless detection data associated with the wireless detection status is “undetected”). In some examples, the wireless detection data includes a flag (or “wireless detection flag”) (or “detection flag”) utilized by the corresponding one of the monitor 108 to indicate whether the wireless device is detected. For instance, the detection flag initially includes a value of “0” prior to any wireless detection; and the monitor 108 sets the detection flag (e.g., modifies the flag to have a value of “1”) in response to the corresponding one of the vibration-sensing devices 102 being detected.

In various implementations, the pairing operations include the monitor 108 pairing with the other wireless device (e.g., any of the vibration-sensing devices 102). In some examples, the pairing operations are performed in response to the detecting a corresponding one of the vibration-sensing devices 102 being performed (e.g., in response to successful completion of the detecting, which indicates a presence of the corresponding one of the vibration-sensing devices 102). In those or other examples, the pairing operations are performed in response to the detection status data indicating “detected.” In those or other examples, the pairing operations are performed in response to the detection flag data being set.

For instance, the pairing operations are utilized to pair the vibration-sensing devices 102 and the monitor 108. In some examples, the pairing is initiated in response to a signal (e.g., a signal transmitted via the wireless protocol) (e.g., a wireless protocol signal) (e.g., a short-range wireless communication protocol signal) (e.g., a pairing request) transmitted by a corresponding one of the vibration-sensing devices 102 and to the monitor 108. In those or other examples, the pairing is initiated in response to the wireless protocol signal) being transmitted by the corresponding one of the vibration-sensing devices 102 and to the monitor 108, in response to the distance (e.g., the distance between the corresponding one of the vibration-sensing devices 102 and the monitor 108) being less than or equal to the threshold distance. In those or other examples, the pairing is initiated in response to the corresponding one of the monitor 108 converting, via the adapter(s) 112, the wireless protocol signal to another signal (e.g., a signal transmitted via the wired protocol) (e.g., a wired protocol signal). For example, the pairing is performed in response to the wired protocol signal being received by the monitor 108 and from the adapter(s) 112.

In some examples, the monitor 108 utilizes the adapter(s) 112 to pair the monitor 108 with the corresponding one of the vibration-sensing devices 102. In those or other examples, pairing (e.g., pairing performed utilizing the pairing operations) is performed between the corresponding one of the vibration-sensing devices 102 and the monitor 108, by performing the pairing operations. In those or other examples, completion of the pairing is achieved in response to completion of the pairing operations.

In various implementations, the pairing includes pairing request values. For example, any of the pairing request values includes an identifier, an authentication flag, or a type of information (e.g., a value identifying the information type). In some cases, the identifier, the authentication flag, the type of information, any other type of pairing request value, or any combination thereof, is transmitted to the monitor 108 and by a pairing device (e.g., the corresponding one of the vibration-sensing devices 102).

In some examples, the monitor 108 compares individual ones of the pairing request values to corresponding pairing characteristics (e.g., corresponding predefined pairing values, corresponding threshold pairing values, etc.). In those or other examples, the monitor 108 identifies whether the pairing is successful in response to the corresponding pairing characteristics being satisfied. The monitor 108, for example, utilizes the pairing request values to perform the pairing in response to the pairing request values satisfying the corresponding pairing characteristics. For example, the pairing is performed in response to the identifier matching an identifier characteristic (e.g., a characteristic indicating an identifier associated with the device (e.g., the vibration-sensing devices 102) enabled, activated, etc., to be paired), the authentication flag having a value matching a flag characteristic (e.g., a characteristic indicating a flag indicating that pairing is allowed, enabled, etc. for the device (e.g., the corresponding one of the vibration-sensing devices 102)), the type of information to be communicated for the device (e.g., the corresponding one of the vibration-sensing devices 102) matching and/or satisfying an information type characteristic (e.g., a characteristic indicating an allowed information type)), or any combination thereof.

In some cases, the monitor 108 performs the pairing operations (e.g., the pairing is performed between the corresponding one of the vibration-sensing devices 102 and the monitor 108) in response to a distance between the adapter(s) 112 and the corresponding one of the vibration-sensing devices 102 being less than a threshold distance (e.g., a maximum distance) (e.g., 4-10 centimeters) (e.g., 4 centimeters, 10 centimeters, 20 centimeters, etc., with a variation of 1 millimeters, 1 centimeter, 2 centimeters, etc.). In various implementations, the pairing includes a tap-to-pair pairing process.

In some instances, the distance between the adapter(s) 112 and the corresponding one of the vibration-sensing devices 102 utilized to initiate (e.g., begin) the pairing is the same as the distance utilized for the performing of the wireless device detecting. Alternatively, the distance between the adapter(s) 112 and the corresponding one of the vibration-sensing devices 102 utilized to initiate (e.g., begin) the pairing is different from the distance utilized for the performing of the wireless device detecting.

In some implementations, pairing data, a pairing flag, etc., is utilized in a similar way as the detection data, the detection flag, etc., respectfully. For example, the pairing data includes a value (e.g., a value of “paired” or “unpaired”), the pairing flag includes a value (e.g., a value of “0” or “1”), or any combination thereof, in response to whether the pairing is successfully completed. For example, the pairing flag (e.g., a pairing request flag) is set in response to the corresponding one of the vibration-sensing devices 102 being authenticated (e.g., authentication via the identifier matching a stored identifier (e.g., the identifier characteristic) being utilized as authentication) via the pairing.

In various implementations, the communicating operations include the monitor 108 communicating with the other wireless device (e.g., the corresponding one of the vibration-sensing devices 102). In some examples, the communicating operations are performed in response to the detecting being performed (e.g., in response to successful completion of the detecting, which indicates a presence of the corresponding one of the vibration-sensing devices 102), and, possibly, in response to the pairing being performed, and, possibly, in response to any other wired/wireless operations being performed. In those or other examples, the communicating operations are performed, possibly, in response to the detection status data indicating “detected,” the detection flag data being set, the pairing status data indicating “paired,” the pairing flag data being set, or any combination thereof.

In various examples, the communicating is performed utilizing security keys. In some cases, the communicating (e.g., communicating of any data transmitted by the corresponding one of the vibration-sensing devices 102, any data transmitted by the monitor 108, or any combination thereof) includes the corresponding one of the vibration-sensing devices 102 transmitting a security key to the monitor 108, via the adapter(s) 112, and the monitor 108 receiving the wired protocol signal further comprises receiving, via the port, the wired protocol signal comprising a security key provided by the corresponding one of the vibration-sensing devices 102.

In various cases, the communicating is performed in response to a successful comparison being performed by the monitor 108 of the security key (e.g., a received security key). For example, the monitor 108 compares the received security key to a stored security (e.g., a predetermined security key, a previously generated security key, a previously received security key, or any combination thereof).

In some cases, the communicating is performed in response to establishing a communication channel (e.g., a secure communication channel). For example, the monitor 108 compares the received security key to the stored security and establishes the communication channel (e.g., a communication channel between the corresponding one of the vibration-sensing devices 102 and the monitor 108, via the adapter(s) 112), in response to a satisfactory result of the comparison. In various cases, the communication channel is established, in response to verifying the security is received security key includes a valid security key, in response to the satisfactory result of the comparison.

For instance, the communicating operations are utilized to exchange wired/wireless communications (e.g., wired/wireless signals utilizing the wired protocol and the wireless protocol) (e.g., USB/NFC signals between the vibration-sensing devices 102 and the monitor 108. In some examples, the monitor 108 utilizes the adapter(s) 112 to exchange communications with the corresponding one of the vibration-sensing devices 102. In those or other examples, communicating (e.g., communicating performed utilizing the communicating operations) is performed between the vibration-sensing devices 102 and the monitor 108, by performing the communicating operations.

In some cases, the monitor 108 performs the communicating operations (e.g., the communicating is performed between the corresponding one of the vibration-sensing devices 102 and the monitor 108) in response to a distance between the adapter(s) 112 and the corresponding one of the vibration-sensing devices 102 being less than a threshold distance (e.g., a maximum distance) (e.g., 4 centimeters, 10 centimeters, 20 centimeters, etc., with a variation of 1 millimeters, 1 centimeter, etc.). In some instances, the distance between the adapter(s) 112 and the corresponding one of the vibration-sensing devices 102 utilized to initiate (e.g., begin), perform, etc., the communicating is the same as the distance utilized for the performing of the wireless device detecting. Alternatively, the distance between the adapter(s) 112 and the corresponding one of the vibration-sensing devices 102 utilized to initiate (e.g., begin), perform, etc., or any combination thereof, the communicating the same as, or different from, the distance utilized for the performing of the wireless device detecting.

In some instances, the distance between the adapter(s) 112 and the corresponding one of the vibration-sensing devices 102 utilized to initiate (e.g., begin), perform, etc., the communicating is the same as the distance utilized for the performing of the wireless device pairing. Alternatively, the distance between the adapter(s) 112 and the corresponding one of the vibration-sensing devices 102 utilized to initiate (e.g., begin), perform, etc., or any combination thereof, the communicating the same as, or different from, the distance utilized for the performing of the wireless device pairing.

In some implementations, communicating data, a communicating flag, etc., is utilized in a similar way as the communicating data, the communicating flag, etc., respectfully. For example, the communicating data includes a value (e.g., a value of “communicated” or “not yet communicated”), the communicating flag includes a value (e.g., a value of “0” or “1”), or any combination thereof, in response to whether the communicating is successfully completed.

In various cases, the communicating data includes data that is transmitted by the monitor 108, via the adapter(s) 112, and to the corresponding one of the vibration-sensing devices 102, data that is transmitted by the corresponding one of the vibration-sensing devices 102 and to the monitor 108, via the adapter, 106, or any combination thereof. Data (e.g., data stored in the monitor 108) that is communicated, via the wired/wireless communications, in some examples, via transmissions to the corresponding one of the vibration-sensing devices 102, is stored in the corresponding one of the vibration-sensing devices 102, such as, for instance, with data being transmitted by the monitor 108, via the adapter(s) 112, and to the corresponding one of the vibration-sensing devices 102. Data (e.g., data stored in the corresponding one of the vibration-sensing devices 102) that is communicated, via the wired/wireless communications, in some examples, via transmissions to the monitor 108, is stored in the monitor 108, such as, for instance, with data being transmitted by the corresponding one of the vibration-sensing devices 102, and to the monitor 108, via the adapter(s) 112.

In various cases, the wired/wireless operations are utilized for various purposes. For example, the wired/wireless operations are utilized as part of, to support, to enable, etc., an ECG functionality (e.g., an ECG mode), a blood oxygenation functionality (e.g., a blood oxygenation mode), a capnography functionality (e.g., a capnography functionality mode), an invasive blood pressure (IBP) functionality (e.g., IBP mode), a non-invasive blood pressure functionality (e.g., a non-invasive blood pressure mode), a mechanical cardio pulmonary respiratory (CPR) functionality (e.g., a CPR mode), a logistical functionality (e.g., a logistical mode), a location services functionality (e.g., a location mode), a CPR feedback functionality (e.g., a CPR feedback mode), an ultrasound functionality (e.g., a ultrasound mode), a video laryngoscope functionality (e.g., a video laryngoscope mode), a payment system functionality (e.g., a payment system mode) (or “payment mode”), an inventory system functionality (e.g., an inventory system mode) (or “inventory mode”), a record keeping system functionality (e.g., a record keeping system mode) (or “record keeping mode”), a smart phone functionality (e.g., a smart phone (e.g., mobile device) (e.g., cellular device) mode), an employee badge functionality (e.g., an employee badge mode), or an RF asset tracking tag functionality (e.g., an RF asset tracking tag mode), any other medical functionality, any other non-medical functionality, or any combination thereof.

In some examples, the wired/wireless operations are utilized as part of, to support, to enable, etc., ECG sensors, blood oxygenation sensors, capnography sensors, invasive blood pressure (IBP) sensors, non-invasive blood pressure sensors, mechanical CPR sensors, logistical sensors, location sensors, CPR feedback sensors, ultrasound sensors, video laryngoscope sensors, payment system sensors, smart phone sensors, employee badge sensors, RF asset tracking tag sensors, any other medical sensors, any other non-medical sensors, or any combination thereof. In those or other examples, the wired/wireless operations are utilized as part of, to support, to enable, etc., ECG devices, blood oxygenation devices, capnography devices, invasive blood pressure (IBP) devices, non-invasive blood pressure devices, mechanical CPR devices, logistical devices, location devices, CPR feedback devices, ultrasound devices, video laryngoscope devices, payment system devices, inventory devices, smart phone devices, employee badge devices, RF asset tracking tag devices, any other medical devices, any other non-medical devices, or any combination thereof.

While the sensors are implemented differently from the devices associated with the wired/wireless operations, in some cases, as discussed above in the current disclosure, it is not limited as such. In some examples, any of the sensors (e.g., any of the sensors associated with the wired/wireless operations) are part of, combined with, integrated together with, etc., in any way, any of the devices (e.g., any of the devices associated with the wired/wireless operations) (e.g., any of the vibration-sensing device(s) 102).

In some cases, any of various operations (e.g., any of the monitor and/or adapter operations, any of the wired/wireless operations, any other operations of any device, such as the monitor 108, the communications connection device(s) 110, and/or the adapter(s) 112, a wireless field associated with any of the vibration-sensing devices 102, the communications connection device(s) 110, or any combination thereof) is performed in response to a user selection, or combination of user selections, received via user input to the monitor 108 (e.g., a UI, a graphical user interface (GUI), an input device, etc., or any combination thereof, of the monitor 108). In some cases, the user selections are received via touchscreens, button, keyboards, mice, any other device of the monitor 108, or any combination thereof.

In various examples, the user selections are received in response to a notification or a combination of notifications, output via the monitor 108 (e.g., a display, a touchscreen, the UI, the GUI, etc., or any combination thereof. In some cases, the notifications are output audibly, visually, haptically, etc., or any combination thereof. In various instances, the notifications are output at beginnings of any of the operations to receive user input to proceed, endings of any of the operations to receive user input as confirmation, any intervening portions (e.g., portions between the beginnings and the endings of the operations) to request confirmation to continue or acknowledgement of errors, etc., or any combination thereof.

For instance, the user selections include an adapter configuration selection, an adapter initialization selection, a setup selection, a driver identity, load, stored, and/or install selection, an adapter activation selection, a detection selection, a pair selection, a communication selection, or any combination thereof. In some cases, any of the selections are received in response to corresponding notifications (e.g., corresponding prompts) output by the monitor 108, the notifications requesting the user selections (e.g., confirmation, permission, acknowledgement, etc.) to continue with corresponding operations.

For example, in response to the identified electrical characteristics associated with the adapter(s) 112, a notification is output by the monitor 108 requesting a user selection to proceed with activating the adapter(s) 112. In some instances, the user selection is received via user input as confirmation to proceed with the activating of the adapter(s) 112, and the activating of the adapter(s) 112 is performed by the monitor 108 in response to the user selection. In such instances or other instances, the activating of the adapter(s) 112 is performed in response to power received from the communication connection device(s) 110.

In another example, in response to the converting, the detecting, the pairing, the activating, etc., a notification is output by the monitor 108 requesting a user selection to proceed with establishing a communication channel. In some instances, the user selection is received via user input as confirmation to proceed with the establishing of the communication channel, and the communication channel is established by the monitor 108 in response to the user selection.

In another example, in response to the converting, the detecting, the pairing is performed utilizing communications (e.g., first communications, which include wireless communications received from any of the vibration-sensing devices 102) being converted by the monitor 108, via the adapter(s) 112, to converted communications (e.g., wired communications) associated with the wired protocol. In some cases, the monitor 108 outputs a notification requesting a user selection to proceed with communicating data between a corresponding one of the vibration-sensing devices 102 and the monitor 108.

In some examples, the notification requesting the user selection to proceed with communicating is output in response to a pairing request flag. In those or other examples, the pairing request flag is set in response to a pairing request characteristic (or “pairing characteristic”) being satisfied by a pairing request value. In those or other examples, the notification being output in response to the pairing request flag is utilized to receive a user selection via user input to the monitor 108. In those or other examples, the notification confirms the corresponding one of the vibration-sensing devices 102 is authenticated, in response to the pairing request flag being set.

In those or other examples, in response to the pairing request flag (e.g., the pairing request flag being set) and the user selection (e.g., the user selection being identified, determined, received, etc., or any combination thereof), the communicating (e.g., exchanging the communications (e.g., second communications) is performed. In various cases, the user selection is received via user input to the UI of the monitor 108, the user selection identifying the notification confirming the wireless device is authenticated.

In various cases, the monitor 108 outputs a notification (or “unauthenticated notification”) (e.g., a warning notification) (e.g., a caution notification) in response to individual ones of the pairing request values not matching the other pairing request values (e.g., the predetermined pairing request values). For example, the unauthenticated notification is output (e.g., output such as by the display) to inform a user (e.g., a medical provider) that the corresponding one of the vibration-sensing devices 102 is not authenticated.

In various cases, the corresponding one of the vibration-sensing devices 102 not being authenticated is in response to any of the pairing request values not satisfying the corresponding pairing characteristics, such as for instances in which the corresponding one of the vibration-sensing devices 102 includes a brand, a type, a model, or any combination thereof, that is not associated with any of the pairing request values. For example, the corresponding one of the vibration-sensing devices 102 not being authenticated is in response to the monitor 108 identifying an absence of certification associated with the corresponding one of the vibration-sensing devices 102.

In some cases, with any of the pairing request values not being satisfied, such as for instance with the unauthenticated notification being output, or for instance with no unauthenticated notification being output, the monitor 108 prevents, blocks, or any combination thereof, some or all of the wired/wireless operations (e.g., the communicating) for the corresponding one of the vibration-sensing devices 102. For example, the monitor 108 prevents, blocks, or any combination thereof, performance of some or all of the wired/wireless operations (e.g., the communicating) with respect to the corresponding one of the vibration-sensing devices 102, with or without the unauthenticated notification being output.

In various implementations, the monitor 108 receives user selections (e.g., an override selection) associated with user input to the monitor 108, in response to the authentication notification. For example, the monitor 108 overrides the preventing of some or all of the wired/wireless operations (e.g., the communicating) for the corresponding one of the vibration-sensing devices 102, in response to the override user selection. Alternatively, the monitor 108 refrains (e.g., such as according to one or more initial values, and/or one or more user input-based values, of one or more settings of the monitor 108) from accepting any override selections and/or disables processing of any user input (e.g., thereby unalterably/automatically controlling the performance, as discussed above, based on the pairing request values not being satisfied, the unauthenticated notification being output, or any combination thereof).

In some instances, by utilizing devices (e.g., the monitor 108), with adapters (e.g., the adapter(s) 112) capable of being utilized to perform the wired/wireless conversions (e.g., the USB/NFC conversions), communications are exchanged via the USB and NFC protocols in a faster and more secure way than by utilizing other protocols (e.g., a BLUETOOTH™ protocol). For example, by utilizing the NFC protocol, cross talk that otherwise may occur while utilizing the BLUETOOTH™ protocol is reduced, prevented, and/or eliminated. In some cases, triage situations with multiple devices that utilize the BLUETOOTH™ protocol and that unintentionally “try to pair together” due to one of the devices being booted up are reduced, prevented, and/or eliminated by utilizing the adapters that are capable of being utilized to perform the USB/NFC conversions.

In some cases, by utilizing the adapters that are capable of being utilized to perform the wired/wireless conversions (e.g., the USB/NFC conversions), cables that may otherwise require wired connections to the monitor 108 are able to be avoided and replaced with cables communicating wirelessly via the adapters. By avoiding cables with wired connections, cable management is unnecessary, which increases safety, efficiency, and effectiveness of medical treatment being provide to subjects via devices utilizing communications via the adapters that are capable of being utilized to perform the USB/NFC conversions. As a result, numbers of successful medical treatments are increased, numbers of lives being saved are increased, and opportunities for occurrences of problems are decreased.

By utilizing the adapters that are capable of being utilized to perform the wired/wireless conversions, transporting and monitoring of subjects being treated utilizing the devices with the adapters are simpler and more streamlined. By utilizing the adapters that are capable of being utilized to perform the wired/wireless conversions, various devices that are not otherwise compatible with the NFC protocol are able to communicate via the NFC protocol, in response to the wired/wireless conversions (e.g., the USB/NFC conversions) being performed utilizing the adapters. Cables being paired (e.g., wirelessly paired) to the devices with the adapters, for instance, are able to be quickly paired, such as by tap-to-pairing processes, regardless of the devices not being capable of performing the wired/wireless conversions without the adapters, in response to the devices and the adapters being coupled together (e.g., being connected or integrated together).

In some cases, alternatively or additionally to the communications connection device(s) 110 including the port as discussed above, any of the communications connection device(s) 110 includes a plug. In some cases, alternatively or additionally to the communication connection device(s) in the adapter(s) 112 including the plug, any of the communication connection device(s) in the adapter(s) 112 includes a port.

In some examples, alternatively or additionally to the communications connection device(s) 110 including the wired transceiver, as discussed above, the communications connection device(s) 110 includes a transceiver (e.g., a transceiver including one or more antennas), such as a wireless communication transceiver (or “wireless transceiver”). In those or other examples, the wireless transceiver included in the communications connection device(s) 110 is compatible with a wireless communications protocol (or “wireless protocol”) (e.g., an RF communication protocol) (e.g., a short-range communications protocol). For instance, the other wireless transceiver included in the communications connection device(s) 110 includes a near field communication (NFC) transceiver (e.g., a transceiver compatible with an NFC protocol). However, in another instance, the wireless transceiver included in the communications connection device(s) 110 includes a BLUETOOTH™ transceiver (e.g., a transceiver compatible with a BLUETOOTH™ protocol), a BLUETOOTH™ low energy (BLE) transceiver (e.g., a transceiver compatible with a BLE protocol), an ultrasonic transceiver (e.g., a transceiver compatible with a ultrasonic protocol), a ZIGBEE® transceiver (e.g., a transceiver compatible with an ZIGBEE® protocol), a wi-fi transceiver (e.g., a transceiver compatible with an a wi-fi protocol), or an institute of electrical and electronics engineers (IEEE) 802.15.4 (e.g., a transceiver compatible with an IEEE 802.15.4 protocol) transceiver, a z-wave transceiver (e.g., a transceiver compatible with a z-wave protocol), or any other type of wireless transceiver (e.g., a transceiver compatible with any other type of wireless communication protocol).

FIGS. 2A-2D illustrates example vibration-sensing devices configured to gather and transmit, to adapters, signals with different types of sensor data, including heartbeat data, breath data, NIBP data, and gastrointestinal data. In FIG. 2A, for example, a heartbeat vibration sensing-capable electronic device (also referred to herein simply as “vibration-sensing device”) 202 includes a device circuit 204. In some cases, the vibration-sensing device 202 is communicatively coupled to an adapter 206, which includes a heartbeat data signals routing-capable circuit (also referred to herein simply as “circuit”) 208. In various examples, the vibration-sensing device 202 is utilized to implement the vibration-sensing device 102(A), as discussed above with reference to FIG. 1. In various examples, the adapter 206 is utilized to implement the any of the adapter(s) 112, as discussed above with reference to FIG. 1.

In some cases, the circuit 204 includes one or more sensors utilized to sense vibration associated with a heartbeat (e.g., of any of the subject(s) 106, as discussed above with reference to FIG. 1. For instance, the sensor(s) in the circuit 204 are utilized to generate vibration-based heartbeat data in response to sensing the vibration associated with the heartbeat. In various examples, the circuit 204 includes one or more communications connection devices (e.g., the communication connection device(s) 104(A), as discussed above with reference to FIG. 1). For instance, the communication connection device(s) 104(A) include one or more transceivers utilized to transmit (e.g., to the monitor 108, as discussed above with reference to FIG. 1) one or more signals including the vibration-based heartbeat data.

In various examples, the vibration-based heartbeat data generated by the circuit 204 is utilized to determine whether the vibration-based heartbeat data includes one or more vibration-based heartbeat characteristics. For instance, the determining of whether the vibration-based heartbeat data includes the vibration-based heartbeat characteristic(s) is performed by the vibration-sensing device 202.

In various cases, the vibration-sensing device 202 is utilized to identify whether one or more artifacts exist (e.g., occur and/or are present during sensing of the vibration). In some examples, in response to identifying the artifact(s) exist, the vibration-sensing device 202 performs any of the action(s), as discussed above with reference to FIG. 1. In those or other examples, in response to identifying, by the vibration-sensing device 202, that there are no artifacts (e.g., interfering with sensing of the vibration-sensing device 202), the vibration-sensing device 202 proceeds with identifying the vibration data and/or the vibration characteristic(s) (e.g., the parameter(s)).

In various examples, the vibration-sensing device 202 includes a sensor utilized to detect vibration of a type corresponding to a heartbeat of a subject 106. In some cases, the sensor detects any types of vibration, such as the heartbeat, a breath, an NIBP, a gastrointestinal function, and/or one or more other types of activity/characteristics. In some instances, the vibration-sensing device 202 filters vibration to identify one or more portions of the vibration associated with the heartbeat. In some examples, the vibration-sensing device 202 generates the vibration-based heartbeat data (e.g., the vibration-based heartbeat characteristic(s)) in response to the filtering of the vibration. For example, the vibration-based heartbeat data corresponds to the portion(s) of the vibration associated with the heartbeat, in response to filtering out, by the vibration-sensing device 202 one or more portions (e.g., one or more remaining portions) of the vibration not associated with the heartbeat.

In various implementations, the vibration-sensing device 202 senses vibration and generates the vibration data. In various instances, the vibration-sensing device 202, in response to the generating of the vibration data, filters the vibration data to identify one or more portions of the vibration data, as the vibration-based heartbeat data. For instance, the vibration-sensing device 202 filters out one or more portions (e.g., one or more remaining portions) of the vibration data not associated with the heartbeat. In some cases, the vibration-sensing device 202 identifies the vibration-based heartbeat data in response to the filtering out of the portion(s) of the vibration data not associated with the heartbeat.

In some cases, individual ones of the remaining portion(s) of the vibration not associated with the heartbeat are associated with a physiological activity/characteristic and/or a different type of activity/characteristic. In some examples, the vibration-sensing device 202 generates the vibration data by utilizing the circuit 204 and/or a processor of the vibration-sensing device 202 to perform the filtering.

In various examples, at least one portion (e.g., a partial portion or an entire portion) of any of the filtering is performed by the vibration-sensing device 202. In alternative examples, at least one portion (e.g., a partial portion or an entire portion) of any of the filtering is performed by the monitor 108.

In FIG. 2B, for example, a breath vibration sensing-capable vibration-sensing device (also referred to herein simply as “vibration-sensing device”) 210 includes a device circuit 212. In some cases, the vibration-sensing device 210 is communicatively coupled to an adapter 214, which includes a breath data signals routing-capable circuit (also referred to herein simply as “circuit”) 216. In various examples, the vibration-sensing device 210 is utilized to implement the vibration-sensing device 102(B), as discussed above with reference to FIG. 1. In various examples, the adapter 214 is utilized to implement the any of the adapter(s) 112, as discussed above with reference to FIG. 1.

In some cases, the circuit 212 includes one or more sensors utilized to sense vibration associated with a breath (e.g., of any of the subject(s) 106, as discussed above with reference to FIG. 1. For instance, the sensor(s) in the circuit 212 are utilized to generate vibration-based breath data in response to sensing the vibration associated with the breath. In various examples, the circuit 212 includes one or more communications connection devices (e.g., the communication connection device(s) 104(B), as discussed above with reference to FIG. 1). For instance, the communication connection device(s) 104(B) include one or more transceivers utilized to transmit (e.g., to the monitor 108) one or more signals including the vibration-based breath data.

In various examples, the vibration-based breath data generated by the circuit 212 is utilized to determine whether the vibration-based breath data includes one or more vibration-based breath characteristics. For instance, the determining of whether the vibration-based breath data includes the vibration-based breath characteristic(s) is performed by the vibration-sensing device 202 and/or the monitor 108.

In various cases, the vibration-sensing device 210 is utilized to identify whether one or more artifacts exist (e.g., occur and/or are present during sensing of the vibration). In some examples, in response to identifying the artifact(s) exist, the vibration-sensing device 210 performs any of the action(s), as discussed above with reference to FIG. 1. In those or other examples, in response to identifying, by the vibration-sensing device 210, that there are no artifacts (e.g., interfering with sensing of the vibration-sensing device 210), the vibration-sensing device 210 proceeds with identifying the vibration data and/or the vibration characteristic(s) (e.g., the parameter(s)).

In various cases, the device circuit 212 is the same type as, or a different type from, the device circuit 204; and/or the circuit 216 is the same type as, or a different type from, the circuit 208. For example, a type of circuit is characterized by types and/or numbers of components included in the circuit, types and/or numbers of circuits included in the circuit, types and/or numbers of functions performed by the circuit, and so on, or any combination thereof.

In various examples, the vibration-sensing device 210 includes a sensor utilized to detect vibration of a type corresponding to a breath of a subject 106. In some cases, the sensor detects any types of vibration, such as a heartbeat, the breath, an NIBP, a gastrointestinal function, and/or one or more other types of activity/characteristics. In some instances, the vibration-sensing device 210 filters vibration to identify one or more portions of the vibration associated with the breath. In some examples, the vibration-sensing device 210 generates the vibration-based breath data (e.g., the vibration-based breath characteristic(s)) in response to the filtering of the vibration. For example, the vibration-based breath data corresponds to the portion(s) of the vibration associated with the breath, in response to filtering out, by the vibration-sensing device 210 one or more portions (e.g., one or more remaining portions) of the vibration not associated with the breath.

In various implementations, the vibration-sensing device 210 senses vibration and generates the vibration data. In various instances, the vibration-sensing device 210, in response to the generating of the vibration data, filters the vibration data to identify one or more portions of the vibration data, as the vibration-based breath data. For instance, the vibration-sensing device 210 filters out one or more portions (e.g., one or more remaining portions) of the vibration data not associated with the breath. In some cases, the vibration-sensing device 210 identifies the vibration-based breath data in response to the filtering out of the portion(s) of the vibration data not associated with the breath.

In some cases, individual ones of the remaining portion(s) of the vibration not associated with the breath are associated with a physiological activity/characteristic and/or a different type of activity/characteristic. In some examples, the vibration-sensing device 210 generates the vibration data by utilizing the circuit 212 and/or a processor of the vibration-sensing device 210 to perform the filtering.

In various examples, at least one portion (e.g., a partial portion or an entire portion) of any of the filtering is performed by the vibration-sensing device 210. In alternative examples, at least one portion (e.g., a partial portion or an entire portion) of any of the filtering is performed by the monitor 108.

In FIG. 2C, for example, an NIBP vibration sensing-capable vibration-sensing device (also referred to herein simply as “vibration-sensing device”) 218 includes a device circuit 220. In some cases, the vibration-sensing device 218 is communicatively coupled to an adapter 222, which includes an NIBP data signals routing-capable circuit (also referred to herein simply as “circuit”) 224. In various examples, the vibration-sensing device 210 is utilized to implement the vibration-sensing device 102(C), as discussed above with reference to FIG. 1. In various examples, the adapter 222 is utilized to implement the any of the adapter(s) 112, as discussed above with reference to FIG. 1.

In some cases, the circuit 220 includes one or more sensors utilized to sense vibration associated with an NIBP (e.g., of any of the subject(s) 106, as discussed above with reference to FIG. 1. For instance, the sensor(s) in the circuit 220 are utilized to generate vibration-based NIBP data in response to sensing the vibration associated with the NIBP. In various examples, the circuit 220 includes one or more communications connection devices (e.g., the communication connection device(s) 104(C), as discussed above with reference to FIG. 1). For instance, the communication connection device(s) 104(C) include one or more transceivers utilized to transmit (e.g., to the monitor 108) one or more signals including the vibration-based NIBP data.

In various examples, the vibration-based breath data generated by the circuit 220 is utilized to determine whether the vibration-based NIBP data includes one or more vibration-based NIBP characteristics. For instance, the determining of whether the vibration-based NIBP data includes the vibration-based NIBP characteristic(s) is performed by the vibration-sensing device 202 and/or the monitor 108.

In various cases, the vibration-sensing device 218 is utilized to identify whether one or more artifacts exist (e.g., occur and/or are present during sensing of the vibration). In some examples, in response to identifying the artifact(s) exist, the vibration-sensing device 218 performs any of the action(s), as discussed above with reference to FIG. 1. In those or other examples, in response to identifying, by the vibration-sensing device 218, that there are no artifacts (e.g., interfering with sensing of the vibration-sensing device 218), the vibration-sensing device 218 proceeds with identifying the vibration data and/or the vibration characteristic(s) (e.g., the parameter(s)).

In various cases, the device circuit 220 is the same type as, or a different type from, the device circuit 204 and/or the device circuit 212. In various cases, the circuit 224 is the same type as, or a different type from, the circuit 208 and/or the circuit 216. For example, a type of circuit is characterized by types and/or numbers of components included in the circuit, types and/or numbers of circuits included in the circuit, types and/or numbers of functions performed by the circuit, and so on, or any combination thereof.

In various examples, the vibration-sensing device 218 includes a sensor utilized to detect vibration of a type corresponding to an NIBP of a subject 106. In some cases, the sensor detects any types of vibration, such as a heartbeat, a breath, the NIBP, a gastrointestinal function, and/or one or more other types of activity/characteristics. In some instances, the vibration-sensing device 218 filters vibration to identify one or more portions of the vibration associated with the NIBP. In some examples, the vibration-sensing device 218 generates the vibration-based NIBP data (e.g., the vibration-based NIBP characteristic(s)) in response to the filtering of the vibration. For example, the vibration-based NIBP data corresponds to the portion(s) of the vibration associated with the NIBP, in response to filtering out, by the vibration-sensing device 218 one or more portions (e.g., one or more remaining portions) of the vibration not associated with the NIBP.

In various implementations, the vibration-sensing device 218 senses vibration and generates the vibration data. In various instances, the vibration-sensing device 218, in response to the generating of the vibration data, filters the vibration data to identify one or more portions of the vibration data, as the vibration-based NIBP data. For instance, the vibration-sensing device 218 filters out one or more portions (e.g., one or more remaining portions) of the vibration data not associated with the NIBP. In some cases, the vibration-sensing device 218 identifies the vibration-based NIBP data in response to the filtering out of the portion(s) of the vibration data not associated with the NIBP.

In some cases, individual ones of the remaining portion(s) of the vibration not associated with the NIBP are associated with a physiological activity/characteristic and/or a different type of activity/characteristic. In some examples, the vibration-sensing device 218 generates the vibration data by utilizing the circuit 220 and/or a processor of the vibration-sensing device 218 to perform the filtering.

In various examples, at least one portion (e.g., a partial portion or an entire portion) of any of the filtering is performed by the vibration-sensing device 202. In alternative examples, at least one portion (e.g., a partial portion or an entire portion) of any of the filtering is performed by the monitor 108.

In FIG. 2D, for example, a gastrointestinal vibration sensing-capable vibration-sensing device (also referred to herein simply as “vibration-sensing device”) 226 includes a device circuit 228. In some cases, the vibration-sensing device 226 is communicatively coupled to an adapter 230, which includes a gastrointestinal data signals routing-capable circuit (also referred to herein simply as “circuit”) 232. In various examples, the vibration-sensing device 226 is utilized to implement the vibration-sensing device 102(D), as discussed above with reference to FIG. 1. In various examples, the adapter 230 is utilized to implement the any of the adapter(s) 112, as discussed above with reference to FIG. 1.

In some cases, the circuit 228 includes one or more sensors utilized to sense vibration associated with one or more gastrointestinal characteristics (e.g., of any of the subject(s) 106, as discussed above with reference to FIG. 1. For instance, the sensor(s) in the circuit 228 are utilized to generate vibration-based gastrointestinal data in response to sensing the vibration associated with the gastrointestinal characteristic(s). In various examples, the circuit 228 includes one or more communications connection devices (e.g., the communication connection device(s) 104(D), as discussed above with reference to FIG. 1). For instance, the communication connection device(s) 104(D) include one or more transceivers utilized to transmit (e.g., to the monitor 108) one or more signals including the vibration-based gastrointestinal data.

In various examples, the vibration-based gastrointestinal data generated by the circuit 228 is utilized to determine whether the vibration-based gastrointestinal data includes the vibration-based gastrointestinal characteristic(s). For instance, the determining of whether the vibration-based gastrointestinal data includes the vibration-based gastrointestinal characteristic(s) is performed by the vibration-sensing device 202 and/or the monitor 108.

In various cases, the vibration-sensing device 226 is utilized to identify whether one or more artifacts exist (e.g., occur and/or are present during sensing of the vibration). In some examples, in response to identifying the artifact(s) exist, the vibration-sensing device 226 performs any of the action(s), as discussed above with reference to FIG. 1. In those or other examples, in response to identifying, by the vibration-sensing device 226, that there are no artifacts (e.g., interfering with sensing of the vibration-sensing device 226), the vibration-sensing device 226 proceeds with identifying the vibration data and/or the vibration characteristic(s) (e.g., the parameter(s)).

In various cases, the device circuit 228 is the same type as, or a different type from, the device circuit 204, the device circuit 212, and/or the device circuit 220. In various cases, the circuit 232 is the same type as, or a different type from, the circuit 208, the circuit 216, and/or the circuit 224. For example, a type of circuit is characterized by types and/or numbers of components included in the circuit, types and/or numbers of circuits included in the circuit, types and/or numbers of functions performed by the circuit, and so on, or any combination thereof.

In various examples, the vibration-sensing device 226 includes a sensor utilized to detect vibration of a type corresponding to a gastrointestinal function of a subject 106. In some cases, the sensor detects any types of vibration, such as a heartbeat, a breath, an NIBP, the gastrointestinal function, and/or one or more other types of activity/characteristics. In some instances, the vibration-sensing device 226 filters vibration to identify one or more portions of the vibration associated with the gastrointestinal function. In some examples, the vibration-sensing device 226 generates the vibration-based gastrointestinal data (e.g., the vibration-based gastrointestinal characteristic(s)) in response to the filtering of the vibration. For example, the vibration-based gastrointestinal data corresponds to the portion(s) of the vibration associated with the gastrointestinal function, in response to filtering out, by the vibration-sensing device 226 one or more portions (e.g., one or more remaining portions) of the vibration not associated with the gastrointestinal function.

In various implementations, the vibration-sensing device 226 senses vibration and generates the vibration data. In various instances, the vibration-sensing device 226, in response to the generating of the vibration data, filters the vibration data to identify one or more portions of the vibration data, as the vibration-based gastrointestinal data. For instance, the vibration-sensing device 226 filters out one or more portions (e.g., one or more remaining portions) of the vibration data not associated with the gastrointestinal function. In some cases, the vibration-sensing device 226 identifies the vibration-based gastrointestinal data in response to the filtering out of the portion(s) of the vibration data not associated with the gastrointestinal function.

In some cases, individual ones of the remaining portion(s) of the vibration not associated with the gastrointestinal function are associated with a physiological activity/characteristic and/or a different type of activity/characteristic. In some examples, the vibration-sensing device 226 generates the vibration data by utilizing the circuit 228 and/or a processor of the vibration-sensing device 226 to perform the filtering.

In various examples, at least one portion (e.g., a partial portion or an entire portion) of any of the filtering is performed by the vibration-sensing device 226. In alternative examples, at least one portion (e.g., a partial portion or an entire portion) of any of the filtering is performed by the monitor 108.

FIG. 3 illustrates an example environment 300 in which a vibration-sensing device senses vibration and communicates signals received by a monitor that identifies a level of ambient sound is above a threshold and generates an alarm. In some examples, the environment 300 includes a vibration-sensing device 302 worn by a subject 304 in a vehicle 306, such as an ambulance. For instance, the vibration-sensing device 302 transmits signals, which are received by the monitor (e.g., a monitor 308). In various cases, the environment 300 includes a caregiver 310 utilizing data generated by the vibration-sensing device 302 and delivering treatment to the subject 304 (e.g., any of the subject(s) 106, as discussed above with respect to FIG. 1). In various examples, the vibration-sensing device 302 and the monitor 308 are utilized to implement any of the vibration-sensing devices 102 and the monitor 108, respectively, as discussed above with respect to FIG. 1.

In various implementations, the vibration-sensing device 302 and/or the monitor 308 are utilized to manage the data generated by the vibration-sensing device 302. For instance, the data includes vibration data generated in response to vibration (e.g., sound) sensed by the vibration-sensing device 302. In some examples, managing the vibration data includes identifying whether one or more characteristics and/or one or more artifacts are included in the vibration data. For instance, individual ones of the characteristic(s) and/or individual ones of the artifact(s) are associated with any of the vibration data (e.g., heartbeat data, breath data, NIBP data, and/or gastrointestinal data).

In various cases, the artifact(s) are utilized to identify whether the environment 300 is suitable for identifying the characteristic(s) in the vibration-sensing data. In some examples, the artifact(s), representing noise (e.g., ambient noise) in the environment 300, are identified and utilized to identify that a level of the noise (e.g., the ambient noise) exceeds a threshold.

In various examples, in response to identifying the artifact(s) and that the level of the noise exceeds the threshold, the managing of the generated vibration data includes performing one or more of various types of actions. For instance, performing the action(s) includes generating one or more alarms indicating that the artifact(s) is identified and that the level of the noise exceeds the threshold. In various cases, performing the action(s) includes waiting, by the vibration-sensing device 302 and/or the monitor 308, to analyze the vibration data. In some examples, the waiting to analyze the vibration data is performed until no artifacts are identified in the vibration data. In alternative or additional examples, the waiting to analyze the vibration data is performed until level of the noise (e.g., the ambient noise) does not exceed the threshold.

In various implementations, the waiting to analyze the vibration data includes waiting to perform analyzing of various types. For instance, the analyzing is performed following ceasing of the waiting. In some cases, to avoid analyzing noise (e.g., unreliable data) in the vibration data, the analyzing is performed following the ceasing of the waiting. In various examples, data being rendered unreliable due to noise associated with the environment 300, such as due to the vehicle 306 traversing a bumpy road, passing noise emitters of various types, etc., is disregarded.

In some cases, various types of the action(s) are performed in response to the ceasing of the waiting. In various examples, the analyzing performed following the ceasing of the waiting includes analyzing the vibration data to identify the characteristic(s). For instance, the vibration data is analyzed to identify the characteristic(s), which are utilizable by the caregiver 310.

In some examples, the action(s) performed in response to the ceasing of the waiting include presenting, via a UI of the vibration-sensing device 302 and/or a UI of the monitor 308, the vibration data and/or the characteristic(s). In those or other examples, the action(s) performed in response to the ceasing of the waiting include transmitting, by a transceiver of the vibration-sensing device 302 and/or a transceiver of the monitor 308, the vibration data and/or the characteristic(s). The transmitted vibration data and/or the transmitted characteristic(s) are received, for example, by the other device. In some examples, the action(s) performed in response to the ceasing of the waiting enable the vibration data and/or the characteristic(s) to be utilized by the caregiver 310.

In various implementations, the vibration data includes a stream of data. For example, the vibration data includes any type and/or format of data in a file. In some cases, the vibration data includes the data in a sound file (or “audio file”). In various examples, the vibration data includes sound data (or “audio data”) encapsulated in any way and/or any format. In some cases, the vibration data is transmitted via one or more communications with sound data. For instance, the vibration data is transmitted via one or more sound communications. In various examples, any of the signals (e.g., any of the communication(s)) utilized to transmit the vibration data includes a secure communication.

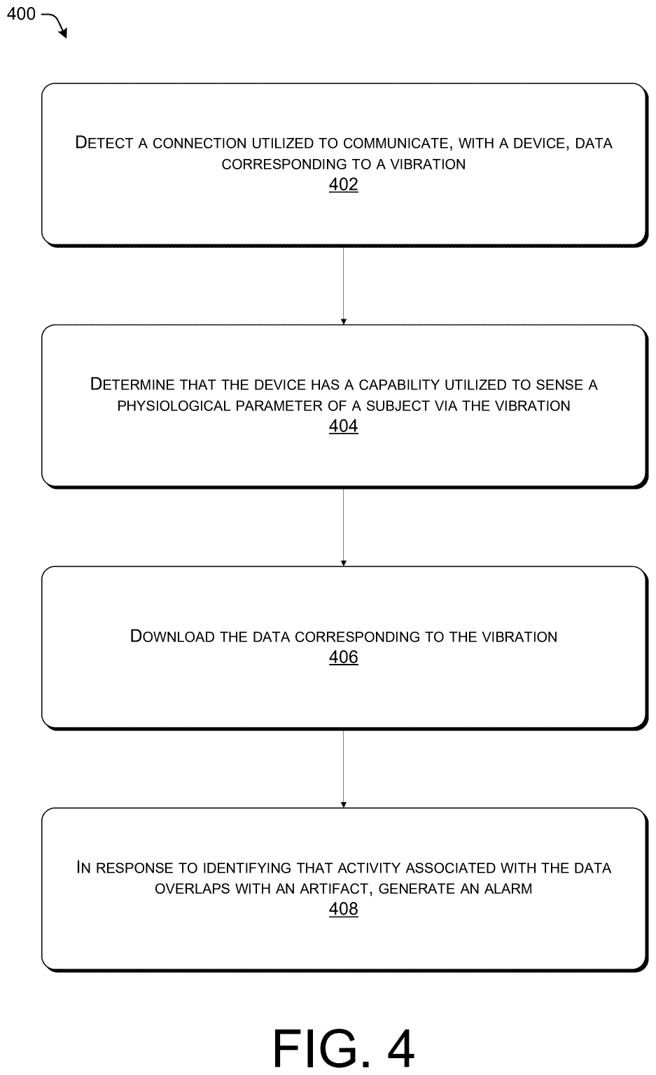

FIG. 4 illustrates an example process 400 for downloading, from devices having capabilities utilized to sense physiological parameters of subjects via vibration, data corresponding to the vibration. The devices for which the signals are exchanged include the vibration-sensing devices 102 and the monitor 108, the vibration-sensing devices 102 being communicatively coupled to the adapter(s) and/or adapter device(s) 112, 206, 214, 222, and 230, as discussed above with reference to FIGS. 1 and 2.

At 402, a medical device detects a connection utilized to communicate, with a device, data corresponding to a vibration. In various cases, the data is received by a connector (e.g., an adapter 112). For instance, the adapter 112 is inserted into a port (e.g., a port of a communications connection device 110) of the medical device (e.g., the monitor 108). In some examples, the adapter 112 is utilized to route (e.g., relay) signals between the device (e.g., a vibration-sensing device 102(A)) and the monitor 108. In some cases, the vibration-sensing device 102(A) are capable of sensing the vibration and generating the data, which includes vibration-based heartbeat data.

At 404, the medical device determines that the device has a capability utilized to sense a physiological parameter of a subject via the vibration. For instance, the medical device (e.g., the monitor 108) determines that the vibration-sensing device 102(A) has the capability utilized to sense the physiological parameter, such as a heartbeat, of the subject (e.g., a subject 106).

At 406, the medical device downloads the data corresponding to the vibration. For instance, the medical device (e.g., the monitor 108) downloads the data in response to determining that the vibration-sensing device 102(A) has the capability utilized to sense the heartbeat of the subject 106.

At 408, the medical device, in response to identifying that activity associated with the data overlaps with an artifact, generates an alarm. For example, the medical device (e.g., the monitor 108) generates the alarm in response to performing a digital handshake with the vibration-sensing device 102(A), downloading the data generated by the vibration, and identifying that the activity associated with the data overlaps with the artifact.

In various examples, the monitor 108 identifies a score analogous to an accuracy with which a characteristic of the heartbeat is aligned with another characteristic of another pattern. For instance, the score being relatively higher represents that the accuracy of the identifying of the characteristic (e.g., a characteristic of a pattern) of the heartbeat is relatively higher. In some cases, the identifying that the activity associated with the data overlaps with the artifact is performed based on identifying that the score is above a threshold. Alternatively, the identifying of the score is performed in response to the identifying that the activity associated with the data overlaps with the artifact.

In some cases, the other characteristic of the other pattern is previously generated by activity that is physical or by activity that is electrical. For instance, the activity includes physical activity being sensed by an accelerometer that is attached to the subject. In such an instance or another instance, the activity includes electrical activity being sensed via a sensor placed against a body of the subject 106.

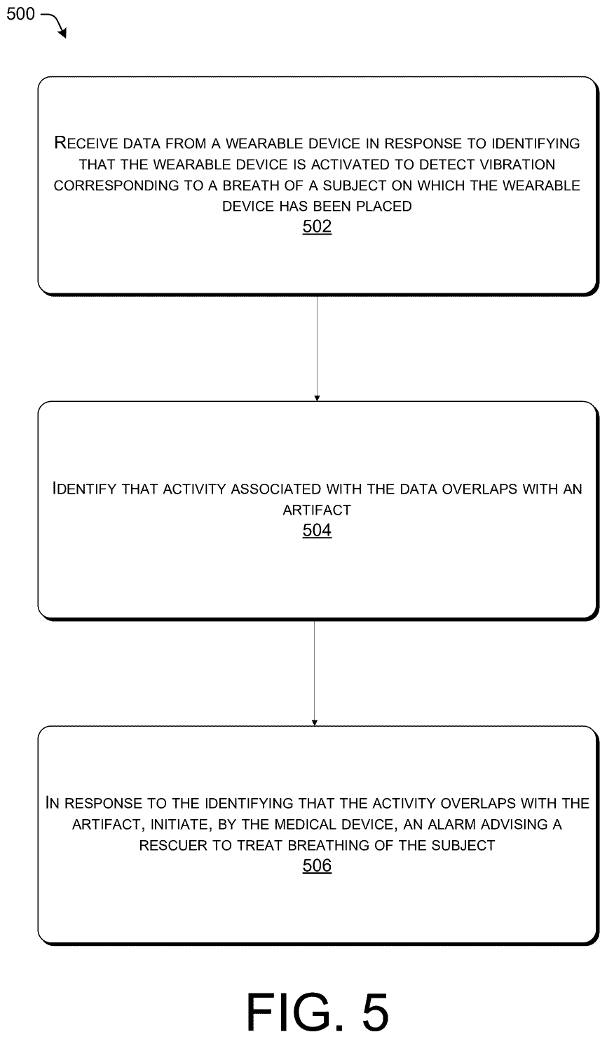

FIG. 5 illustrates an example process 500 for receiving data from wearable devices activated to detect vibration corresponding to breaths of subjects. The devices for which the signals are exchanged include the vibration-sensing devices 102 and the monitor 108, the vibration-sensing devices 102 being communicatively coupled to the adapter device(s) 112, 206, 214, 222, and 230, as discussed above with reference to FIGS. 1 and 2.

At 502, a medical device receives data from a wearable device in response to identifying that the wearable device is activated to detect vibration corresponding to a breath of a subject on which the wearable device has been placed. For example, the medical device (e.g., a monitor 108) receives the data from the wearable device (e.g., a vibration-sensing device 102). In some cases, the data is received in response to identifying that the vibration-sensing device 102 is activated to detect vibration corresponding the breath of the subject (e.g., a subject 106) on which the vibration-sensing device 102 has been placed.

In various cases, the monitor 108 detects a signature routed from the vibration-sensing device 102, and by a connector (e.g., an adapter 112). For instance, the signature is routed by the adapter 112, in response to identifying, by the monitor 108, that the adapter 112 is inserted in a port of the monitor 108. In some examples, the signature is routed by the adapter 112 being activated by power supplied from by the monitor 108. For instance, the monitor 108 detects the signature routed from the wearable device (e.g., the vibration-sensing device 102) and by the adapter 112. In some cases, the adapter 112 is inserted into the port of the medical device (e.g., the monitor 108), and activated by power supplied from the monitor 108.

At 504, the medical device identifies that activity associated with the data overlaps with an artifact. For example, the medical device (e.g., a monitor 108) identifies that the activity associated with the data overlaps with the artifact, such as some type of ambient sound.

In various cases, the monitor 108 receives data from the vibration-sensing device 102 via the adapter 112 in response to the identifying, by the monitor 108, that the vibration-sensing device 102 is activated to detect a vibration corresponding to a breath of a subject on which the vibration-sensing device 102 has been placed. For instance, the data received from the vibration-sensing device 102 includes vibration breath data. In some examples, the vibration breath data is received in response to the monitor 108 identifying that the vibration-sensing device 102 is activated to detect a vibration corresponding to a breath of a subject (e.g., a subject 106) on which the vibration-sensing device 102 has been placed.

At 506, the medical device, in response to the identifying that the activity overlaps with the artifact, initiates an alarm advising a rescuer to treat breathing of the subject. For instance, the medical device (e.g., a monitor 108) initiates and/or generates the alarm advising the rescuer (e.g., a caregiver 310) to treat breathing of the subject (e.g., a subject 106/304).

In some cases, the monitor 108 identifies a pattern of the data that matches, at a level that is greater than a threshold level, another pattern of other data, the other pattern of the other data being associated with activity overlapping with an artifact. For instance, the other pattern of the other data being identified as overlapping with the artifact, is utilized to identify other vibration breath data in the other data is associated with activity overlapping with the artifact.

In various examples, the monitor 108, in response to the identifying of the pattern of the data matches the other pattern of the other data at the level that is greater than the threshold level, identifies that activity associated with the pattern of the data that overlaps with the artifact is associated with vibration breath data. In various instances, the monitor 108 initiates and/or generates the alarm, in response to identifying that the vibration breath data is associated with the activity (e.g., the breath of the subject 106) overlapping with the artifact.

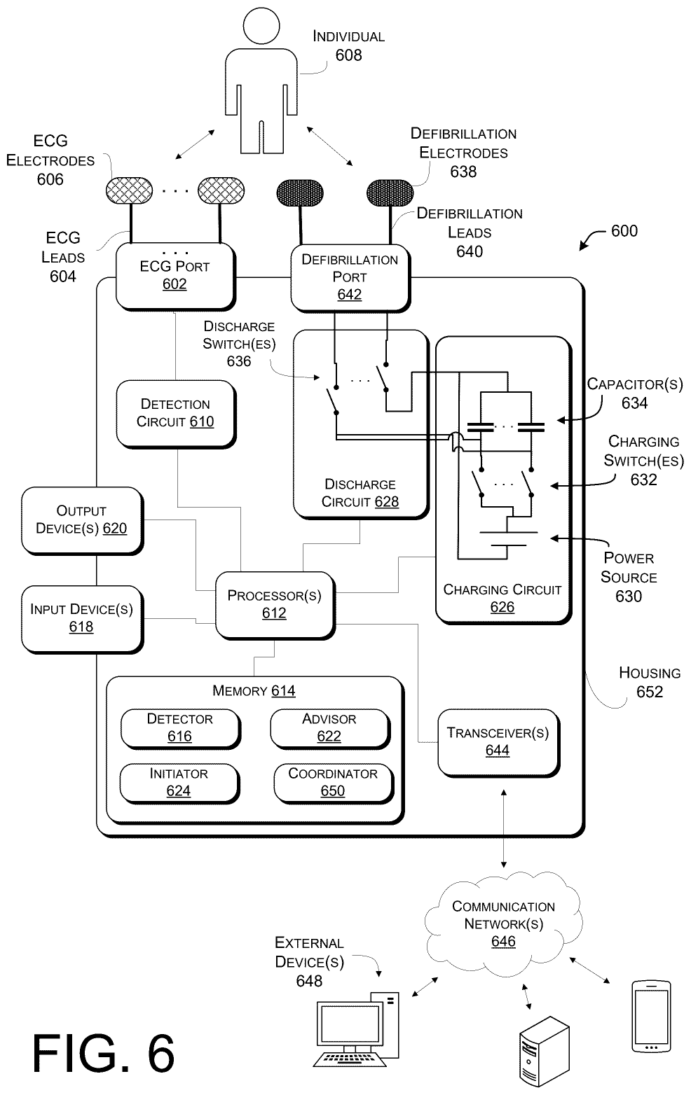

FIG. 6 illustrates an example of an external defibrillator 600 configured to perform various functions described herein. For example, the external defibrillator 600 is utilized to implement the monitor 108 described above with reference to FIGS. 1 to 5.

The external defibrillator 600 includes an ECG port 602 connected to multiple ECG leads 604. In some cases, the ECG leads 604 are removeable from the ECG port 602. For instance, the ECG leads 604 are plugged into the ECG port 602. The ECG leads 604 are connected to ECG electrodes 606, respectively. In various implementations, the ECG electrodes 606 are disposed on different locations on an individual 608. A detection circuit 610 is configured to detect relative voltages between the ECG electrodes 606. These voltages are indicative of the electrical activity of the heart of the individual 608.

In various implementations, the ECG electrodes 606 are in contact with the different locations on the skin of the individual 608. In some examples, a first one of the ECG electrodes 606 is placed on the skin between the heart and right arm of the individual 608, a second one of the ECG electrodes 606 is placed on the skin between the heart and left arm of the individual 608, and a third one of the ECG electrodes 606 is placed on the skin between the heart and a leg (either the left leg or the right leg) of the individual 608. In these examples, the detection circuit 610 is configured to measure the relative voltages between the first, second, and third ECG electrodes 606. Respective pairings of the ECG electrodes 606 are referred to as “leads,” and the voltages between the pairs of ECG electrodes 606 are known as “lead voltages.” In some examples, more than three ECG electrodes 606 are included, such that 6-lead or 12-lead ECG signals are detected by the detection circuit 610.

The detection circuit 610 includes at least one analog circuit, at least one digital circuit, or a combination thereof. The detection circuit 610 receives the analog electrical signals from the ECG electrodes 606, via the ECG port 602 and the ECG leads 604. In some cases, the detection circuit 610 includes one or more analog filters configured to filter noise and/or artifact from the electrical signals. The detection circuit 610 includes an analog-to-digital (ADC) in various examples. The detection circuit 610 generates a digital signal indicative of the analog electrical signals from the ECG electrodes 606. This digital signal can be referred to as an “ECG signal” or an “ECG.”

In some cases, the detection circuit 610 further detects an electrical impedance between at least one pair of the ECG electrodes 606. For example, the detection circuit 610 includes, or otherwise controls, a power source that applies a known voltage (or current) across a pair of the ECG electrodes 606 and detects a resultant current (or voltage) between the pair of the ECG electrodes 606. The impedance is generated based on the applied signal (voltage or current) and the resultant signal (current or voltage). In various cases, the impedance corresponds to respiration of the individual 608, chest compressions performed on the individual 608, and other physiological states of the individual 608. In various examples, the detection circuit 610 includes one or more analog filters configured to filter noise and/or artifact from the resultant signal. The detection circuit 610 generates a digital signal indicative of the impedance using an ADC. This digital signal can be referred to as an “impedance signal” or an “impedance.”

The detection circuit 610 provides the ECG signal and/or the impedance signal one or more processors 612 in the external defibrillator 600. In some implementations, the processor(s) 612 includes a central processing unit (CPU), a graphics processing unit (GPU), both CPU and GPU, or other processing unit or component known in the art.

The processor(s) 612 is operably connected to memory 614. In various implementations, the memory 614 is volatile (such as random access memory (RAM)), non-volatile (such as read only memory (ROM), flash memory, etc.) or some combination of the two. The memory 614 stores instructions that, when executed by the processor(s) 612, causes the processor(s) 612 to perform various operations. In various examples, the memory 614 stores methods, threads, processes, applications, objects, modules, any other sort of executable instruction, or a combination thereof. In some cases, the memory 614 stores files, databases, or a combination thereof. In some examples, the memory 614 includes, but is not limited to, RAM, ROM, electrically erasable programmable read-only memory (EEPROM), flash memory, or any other memory technology. In some examples, the memory 614 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 612 and/or the external defibrillator 600. In some cases, the memory 614 at least temporarily stores the ECG signal and/or the impedance signal.

In various examples, the memory 614 includes a detector 616, which causes the processor(s) 612 to determine, based on the ECG signal and/or the impedance signal, whether the individual 608 is exhibiting a particular heart rhythm. For instance, the processor(s) 612 determines whether the individual 608 is experiencing a shockable rhythm that is treatable by defibrillation. Examples of shockable rhythms include ventricular fibrillation (VF) and ventricular tachycardia (V-Tach). In some examples, the processor(s) 612 determines whether any of a variety of different rhythms (e.g., asystole, sinus rhythm, atrial fibrillation (AF), etc.) are present in the ECG signal.

The processor(s) 612 is operably connected to one or more input devices 618 and one or more output devices 620. Collectively, the input device(s) 618 and the output device(s) 620 function as an interface between a user and the defibrillator 600. The input device(s) 618 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 620 includes at least one of a display, a speaker, a haptic output device, a printer, or any combination thereof. In various examples, the processor(s) 612 causes a display among the input device(s) 618 to visually output a waveform of the ECG signal and/or the impedance signal. In some implementations, the input device(s) 618 includes one or more touch sensors, the output device(s) 620 includes a display screen, and the touch sensor(s) are integrated with the display screen. Thus, in some cases, the external defibrillator 600 includes a touchscreen configured to receive user input signal(s) and visually output physiological parameters, such as the ECG signal and/or the impedance signal.

In some examples, the memory 614 includes an advisor 622, which, when executed by the processor(s) 612, causes the processor(s) 612 to generate advice and/or control the output device(s) 620 to output the advice to a user (e.g., a rescuer). In some examples, the processor(s) 612 provides, or causes the output device(s) 620 to provide, an instruction to perform CPR on the individual 608. In some cases, the processor(s) 612 evaluates, based on the ECG signal, the impedance signal, or other physiological parameters, CPR being performed on the individual 608 and causes the output device(s) 620 to provide feedback about the CPR in the instruction. According to some examples, the processor(s) 612, upon identifying that a shockable rhythm is present in the ECG signal, causes the output device(s) 620 to output an instruction and/or recommendation to administer a defibrillation shock to the individual 608.

The memory 614 also includes an initiator 624 which, when executed by the processor(s) 612, causes the processor(s) 612 to control other elements of the external defibrillator 600 in order to administer a defibrillation shock to the individual 608. In some examples, the processor(s) 612 executing the initiator 624 selectively causes the administration of the defibrillation shock based on determining that the individual 608 is exhibiting the shockable rhythm and/or based on an input from a user (received, e.g., by the input device(s) 618. In some cases, the processor(s) 612 causes the defibrillation shock to be output at a particular time, which is determined by the processor(s) 612 based on the ECG signal and/or the impedance signal.

The processor(s) 612 is operably connected to a charging circuit 626 and a discharge circuit 628. In various implementations, the charging circuit 626 includes a power source 630, one or more charging switches 632, and one or more capacitors 634. The power source 630 includes, for instance, a battery. The processor(s) 612 initiates a defibrillation shock by causing the power source 630 to charge at least one capacitor among the capacitor(s) 634. For example, the processor(s) 612 activates at least one of the charging switch(es) 632 in the charging circuit 626 to complete a first circuit connecting the power source 630 and the capacitor to be charged. Then, the processor(s) 612 causes the discharge circuit 628 to discharge energy stored in the charged capacitor across a pair of defibrillation electrodes 638, which are in contact with the individual 608. For example, the processor(s) 612 deactivates the charging switch(es) 632 completing the first circuit between the capacitor(s) 634 and the power source 630, and activates one or more discharge switches 636 completing a second circuit connecting the charged capacitor 634 and at least a portion of the individual 608 disposed between defibrillation electrodes 638.

The energy is discharged from the defibrillation electrodes 638 in the form of a defibrillation shock. For example, the defibrillation electrodes 638 are connected to the skin of the individual 608 and located at positions on different sides of the heart of the individual 608, such that the defibrillation shock is applied across the heart of the individual 608. The defibrillation shock, in various examples, depolarizes a significant number of heart cells in a short amount of time. The defibrillation shock, for example, interrupts the propagation of the shockable rhythm (e.g., VF or V-Tach) through the heart. In some examples, the defibrillation shock is 200 J or greater with a duration of about 0.016 seconds. In some cases, the defibrillation shock has a multiphasic (e.g., biphasic) waveform. The discharge switch(es) 636 are controlled by the processor(s) 612, for example. In various implementations, the defibrillation electrodes 638 are connected to defibrillation leads 640. The defibrillation leads 640 are connected to a defibrillation port 642, in implementations. According to various examples, the defibrillation leads 640 are removable from the defibrillation port 642. For example, the defibrillation leads 640 are plugged into the defibrillation port 642.

In various implementations, the processor(s) 612 is operably connected to one or more transceivers 644 that transmit and/or receive data over one or more communication networks 646. For example, the transceiver(s) 644 includes a network interface card (NIC), a network adapter, a local area network (LAN) adapter, or a physical, virtual, or logical address to connect to the various external devices and/or systems. In various examples, the transceiver(s) 644 includes any sort of wireless transceivers capable of engaging in wireless communication (e.g., radio frequency (RF) communication). For example, the communication network(s) 646 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 644 includes other wireless modems, such as a modem for engaging in WI-FI®, WIGIG®, WIMAX®, BLUETOOTH®, NFC, radio frequency identification (RFID), or infrared communication over the communication network(s) 646.

The defibrillator 600 is configured to transmit and/or receive data (e.g., ECG data, impedance data, data indicative of one or more detected heart rhythms of the individual 608, data indicative of one or more defibrillation shocks administered to the individual 608, etc.) with one or more external devices 648 via the communication network(s) 646. The external devices 648 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 646. In some examples, the external device(s) 648 is located remotely from the defibrillator 600, such as at a remote clinical environment (e.g., a hospital). According to various implementations, the processor(s) 612 causes the transceiver(s) 644 to transmit data to the external device(s) 648. In some cases, the transceiver(s) 644 receives data from the external device(s) 648 and the transceiver(s) 644 provide the received data to the processor(s) 612 for further analysis.

In some cases, the external device(s) 648 include one or more medical devices. According to various implementations, the memory 614 further includes a coordinator 650 which, when executed by the processor(s) 612, causes the processor(s) 612 to coordinate with the external device(s) 648, such as by administering therapy (e.g., defibrillation, pacing, etc.) to a subject based on communication between the defibrillator 600 and the external device(s) 648, as described herein. In some implementations, the processor(s) 612, when executing the coordinator 650, receives data from the external device(s) 648, analyzes the data to determine a control parameter(s), and administers therapy (e.g., defibrillation, pacing, etc.) in accordance with the control parameter(s), as described herein. In some implementations, the processor(s) 612, when executing the coordinator 650, determines a parameter associated with the therapy being administered by the defibrillator 600, such as a physiological parameter of the individual 608, determines, by analyzing the parameter, a control parameter for controlling administration of therapy to the individual 608 by the external device(s) 648, and sends data (e.g., via the transceiver(s) 644) to the external device(s) 648, the data representing the control parameter. In general, the coordinator 650, when executed by the processor(s) 612, may cause the processor(s) 612 to perform any of the processes 300-800 described herein.

In various implementations, the external defibrillator 600 also includes a housing 652 that at least partially encloses other elements of the external defibrillator 600. For example, the housing 652 encloses the detection circuit 610, the processor(s) 612, the memory 614, the charging circuit 626, the transceiver(s) 644, or any combination thereof. In some cases, the input device(s) 618 and output device(s) 620 extend from an interior space at least partially surrounded by the housing 652 through a wall of the housing 652. In various examples, the housing 652 acts as a barrier to moisture, electrical interference, and/or dust, thereby protecting various components in the external defibrillator 600 from damage.

In some implementations, the external defibrillator 600 is an automated external defibrillator (AED) operated by an untrained user (e.g., a bystander, layperson, etc.) and can be operated in an automatic mode. In automatic mode, the processor(s) 612 automatically identifies a rhythm in the ECG signal, makes a decision whether to administer a defibrillation shock, charges the capacitor(s) 634, discharges the capacitor(s) 634, or any combination thereof. In some cases, the processor(s) 612 controls the output device(s) 620 to output (e.g., display) a simplified user interface to the untrained user. For example, the processor(s) 612 refrains from causing the output device(s) 620 to display a waveform of the ECG signal and/or the impedance signal to the untrained user, in order to simplify operation of the external defibrillator 600.

In some examples, the external defibrillator 600 is a monitor-defibrillator utilized by a trained user (e.g., a clinician, an emergency responder, etc.) and can be operated in a manual mode or the automatic mode. When the external defibrillator 600 operates in manual mode, the processor(s) 612 cause the output device(s) 620 to display a variety of information that may be relevant to the trained user, such as waveforms indicating the ECG data and/or impedance data, notifications about detected heart rhythms, and the like.

### Example Clauses

1: A system, comprising: a wearable device configured to be worn by a subject, the wearable device comprising: a sensor configured to detect a vibration associated with a heartbeat of the subject; and a first transceiver configured to transmit a communication signal including data representing the vibration; and a connector configured to relay signals related to the vibration; a medical device, comprising: a port configured to receive the connector and supply power to the connector, the connector comprising a second transceiver configured to receive data from the first transceiver; a processor configured to: activate the connector; identify the wearable device being positioned at a distance from the connector, the distance being less than or equal to a threshold distance associated with a communication protocol; generate, via the connector, a communication path with the wearable device; receive, via the connector, the data representing the vibration; convert the data to converted data associated with a different protocol; identify a likelihood that the data matches stored data representing a historical vibration that overlaps with an artifact, and that the likelihood is above a threshold likelihood; in response to identifying that the likelihood is above the threshold likelihood, determine that the heartbeat generating the vibration overlaps with the artifact; in response to determining that the heartbeat generating the vibration overlaps with the artifact, generate an alarm; and cause output of the alarm.

2: The system of clause 1, wherein the processor is further configured to: receive, via the connector and using the communication protocol, other data representing another vibration associated with a non-invasive blood pressure (NIBP); analyze the other data being converted by the connector to other converted data associated with the different protocol; identify another likelihood that the other data matches other stored data representing another historical vibration associated with a historical NIBP that overlaps with another artifact, and that the other likelihood is above another threshold likelihood; and in response to identifying that the other likelihood is above the other threshold likelihood, determine that activity associated with the NIBP overlaps with the other artifact.

3: The system of clause 1 or 2, wherein the wearable device comprises an activator configured to trigger the sensor to detect sound via the vibration, the activator configured to trigger the first transceiver to transmit the communication signal.

4: The system of any of clauses 1 to 3, wherein the processor is further configured to, prior to receiving the data: receive, from the wearable device and via the connector, a tag representing that the wearable device has a capability to sense the vibration; and transmit a prompt to the wearable device that triggers the wearable device to transmit the data, wherein receiving the data comprises, in response to the transmitting of the prompt, activating a state of the medical device to receive the data and receiving the data.

5: A medical device, comprising: a transceiver configured to receive data representing vibration identified by a wearable device, the vibration being generated by a heart of a subject emitting a heartbeat; and a processor configured to: determine that the heartbeat identified utilizing the data overlaps with an artifact; and in response to determining that the heartbeat overlaps with the artifact, generate an alarm.

6: The medical device of clause 5, wherein the processor is further configured to: identify a score that corresponds to an accuracy of a characteristic of the heartbeat identified utilizing the data, and that the score is less than a threshold score; in response to the identifying that the score is less than the threshold score, cause the transceiver to transmit a pulse triggering a change in a state of the wearable device to another state of the wearable device that increases an amount of power drawn from a power source by the wearable device; and receive other data from the wearable device operating in the other state.

7: The medical device of clause 5 or 6, wherein the processor is further configured to determine that the artifact overlaps with activity represented by an electrocardiogram (ECG).

8: The medical device of any of clauses 5 to 7, further comprising: a sensor configured to detect a connector being inserted into a port, the connector comprising a capability utilized to relay data representing the vibration, wherein the transceiver is further configured to: exchange codes utilized to pair the medical device with the wearable device via the connector, and, in response to the exchanging of the codes, receive the data.

9: The medical device of any of clauses 5 to 8, wherein the processor is further configured to: identify a characteristic of the heartbeat identified utilizing the data, and that a value of the characteristic is less than a threshold value; in response to the identifying that the value is less than the threshold value, cause the transceiver to transmit a key triggering a change in a state of the wearable device to another state of the wearable device sensing another vibration with a different frequency; and receive other data from the wearable device sensing the other vibration with the different frequency.

10: The medical device of any of clauses 5 to 9, wherein the processor is further configured to: cause the transceiver to transmit a code triggering a change in a state of the wearable device to another state of the wearable device filtering out another vibration with a different frequency; and receive other data from the wearable device filtering out the other vibration with the different frequency.

11: A method, comprising: detecting, by a medical device, a connection utilized to communicate, with a device, data corresponding to a vibration; determining, by the medical device, that the device has a capability utilized to sense a physiological parameter of a subject via the vibration; downloading, by the medical device, the data corresponding to the vibration; and in response to identifying that activity associated with the data overlaps with an artifact, generating an alarm.

12: The method of clause 11, further comprising: identifying that the device is within a distance from the medical device that is less than a threshold; detecting a code being downloaded by a connector that is portable and inserted into a port of the medical device, the code comprising an identifier associated with the device, in response to identifying that the device is within the distance and that the device has the capability utilized to sense a heartbeat, transmitting a tag utilized to pair the medical device with a sensor, via the connector.

13: The method of clause 11 or 12, further comprising: identifying a heartbeat as the physiological parameter; identifying a score analogous to an accuracy with which a characteristic of a pattern of the heartbeat is aligned with another characteristic of another pattern, the other characteristic of the other pattern being previously generated by activity that is physical or by activity that is electrical, the activity that is physical being sensed by an accelerometer that is attached to the subject, the activity that is electrical being sensed via a sensor placed against a body of the subject.

14: The method of any of clauses 11 to 13, further comprising: monitoring whether a level of a characteristic of a heartbeat identified as the physiological parameter is less than a threshold, or whether the level of the characteristic is indeterminate or unclassifiable; and in response to the monitoring, transmitting a trigger causing the device to retransmit data generated by other vibration occurring subsequently to the vibration.

15: The method of any of clauses 11 to 14, further comprising: identifying a gastrointestinal parameter as the physiological parameter; identifying that the artifact overlaps with the activity that is gastrointestinal; and generating the alarm in response to the identifying that the artifact overlaps with the activity that is gastrointestinal.

16: The method of any of clauses 11 to 15, further comprising: identifying a heartbeat as the physiological parameter; identifying that the artifact overlaps with the heartbeat; and generating the alarm in response to the identifying that the artifact overlaps with the heartbeat.

17: The method of any of clauses 11 to 16, further comprising: in response to the downloading of the data, identifying a heartbeat as the physiological parameter, and that the artifact overlaps with the heartbeat; and in response to the identifying the artifact overlaps with the heartbeat, generating the alarm advising a rescuer of a treatment.

18: The method of any of clauses 11 to 17, further comprising: in response to the downloading of the data, identifying a heartbeat as the physiological parameter; identifying that the artifact overlaps with the heartbeat; identifying a weight associated with a treatment representing a likelihood that the treatment will be successful in improving a condition of the subject; and generating an advisory instructing a rescuer to provide the treatment.

19: The method of any of clauses 11 to 18, further comprising: filtering out, from the data generated by the vibration, noise identified as being unrelated to a heartbeat of the subject.

20: The method of any of clauses 11 to 19, further comprising: mapping the data to individual occurrences in historical data identifying patterns of corresponding heartbeats; identifying that a likelihood of a match between the data and an occurrence in the historical data is above a threshold; identifying that the occurrence in the historical data does not include any unexpected patterns; and refraining from generating an advisory representing a heartbeat of the subject in response to the identifying that the occurrence in the historical data does not include any unexpected patterns.

21: A system, comprising: a wearable device configured to be worn by a subject, the wearable device comprising: a sensor configured to detect vibration associated with a breath of the subject; and a first transceiver configured to transmit a communication signal including data representing the vibration; and a connector configured to relay signals related to the vibration; a medical device, comprising: a port configured to receive the connector and supply power to the connector, the connector comprising a second transceiver configured to receive the data from the first transceiver; a processor configured to: generate, via the connector, a communication path with the wearable device being positioned at a distance from the connector that is less than or equal to a threshold distance; utilize the connector to convert initial signals received from the wearable device to the signals related to the vibration, the signals being relayed by the connector according to a different protocol; in response to utilizing the connector to convert the initial signals, identify the signals being relayed by the connector and to the medical device according to the different protocol; receive, via the connector, the data representing the vibration; in response to the receiving of the data representing the vibration, determine the breath that generates the vibration represented by the data overlaps with an artifact; and cause output of an alarm.

22: The system of clause 21, wherein the processor is further configured to: in response to identifying the artifact, identify a classification associated with a historical artifact matching the artifact by a score being above a threshold score; in response to the identifying of the classification associated with the historical artifact, identify that the classification is associated with the artifact; and identify an index representing the alarm, the index being associated with the classification; in response to the identifying of the index, output the alarm; and cease output of the alarm in response to receiving other data relayed by the connector, the other data representing another vibration associated with the breath of the subject.

23: The system of clause 21 or 22, the artifact overlapping with activity associated with a non-invasive blood pressure (NIBP), wherein the processor is further configured to: in response to identifying the artifact, identify a classification of a historical treatment associated with a historical artifact matching the artifact by a score being above a threshold score; in response to the identifying of the classification, identify the historical treatment as a treatment associated with the artifact; and identify an index representing an alarm associated with the treatment; in response to the generating of the index, output the alarm; and cease output of the alarm in response to receiving other data relayed by the connector, the other data representing another vibration associated with the breath of the subject.

24: The system of any of clauses 21 to 23, the artifact overlapping with activity represented by an electrocardiogram (ECG) or a non-invasive blood pressure (NIBP), wherein generating the communication path comprises: outputting a notifier to a rescuer of an availability of the connector; and in response to outputting the notifier and identifying the connector being inserted into the port, generating, via the connector, the communication path; wherein the processor is further configured to: cease output of the alarm in response to receiving other data relayed by the connector, the other data representing another vibration associated with the breath of the subject.

25: A medical device, comprising: a transceiver configured to receive, from a wearable device, data representing vibration identified by the wearable device, the vibration being associated with a breath of a subject; and a processor configured to: determine, by analyzing the vibration, that the breath is indicative of a parameter being less than a threshold parameter; and in response to determining that the data is indicative of the parameter being less than the threshold parameter, initiate an alarm.

26: The medical device of clause 25, wherein the determining that the breath is indicative of the parameter being less than the threshold parameter comprises: outputting a notifier to a rescuer of an availability of a connector that enables the wearable device to be utilized as a substitute for a stethoscope; and in response to outputting the notifier and identifying the connector being inserted into a port, generating, via the connector, a communication path between the wearable device and the medical device.

27: The medical device of clause 25 or 26, the parameter comprising an electrocardiogram (ECG) parameter, wherein outputting the alarm comprises: identifying a historical ECG parameter matching the ECG parameter by a score being above a threshold score; in response to the identifying of the historical ECG parameter, identifying a classification of treatment that is associated with the ECG parameter; and in response to the identifying of the classification, outputting the alarm.

28: The medical device of clause 26, the parameter comprising a non-invasive blood pressure (NIBP) parameter, wherein outputting the alarm comprises: identifying a historical NIBP parameter matching the NIBP parameter by a score being less than a threshold score; in response to the identifying of the historical NIBP parameter, identifying a classification of treatment that is associated with the NIBP parameter; and in response to the identifying of the classification, outputting the alarm.

29: The medical device of any of clauses 25 to 28, wherein receiving the data representing the vibration comprises: detecting, by an activator of a connector inserted into a port, a tap of the wearable device; receiving a prompt from the wearable device being activated by the tap; transmitting an acknowledgement to the wearable device; and receiving the data in response to receiving the prompt and transmitting the acknowledgement.

30: The medical device of any of clauses 25 to 29, further comprising: a sensor configured to generate a signal associated with a connector being inserted into a port, the connector comprising a capability utilized to relay the data received from the wearable device, the wearable device being activated by input received to a user interface (UI), wherein the processor is further configured to: detect the connector being inserted into the port in response to receiving the signal from the sensor; and pair the medical device with the wearable device, via the connector, by causing the transceiver to exchange a code utilized to set up a channel available for communications to be exchanged between the medical device and the wearable device, via the connector.

31: The medical device of any of clauses 25 to 30, wherein the transceiver is further configured to receive, via a connector inserted into a port, the data, the receiving of the data ceasing when the connector is disconnected from the port.

32: A method, comprising: receiving, by a medical device, data from a wearable device in response to identifying that the wearable device is activated to detect vibration corresponding to a breath of a subject on which the wearable device has been placed; identifying that activity associated with the data overlaps with an artifact; and in response to the identifying that the activity overlaps with the artifact, initiating, by the medical device, an alarm advising a rescuer to treat breathing of the subject.

33: The method of clause 32, further comprising: identifying a pattern of new data that matches, at a level that is greater than a threshold level, another pattern of other data, the other pattern of the other data being associated with activity overlapping with an artifact; and in response to the identifying of the pattern that matches the other pattern, identifying the pattern is associated with an electrocardiogram (ECG) parameter.

34: The method of clause 32 or 33, further comprising: identifying a pattern of new data; and identifying the pattern is associated with a non-invasive blood pressure (NIBP) parameter.

35: The method of any of clauses 32 to 34, further comprising: detecting a signature routed from the wearable device and by a connector, in response to identifying the connector being inserted in a port of a medical device and being activated by power supplied by the medical device; in response to the detecting of the signature, identify to change the medical device from a state to not analyze the vibration corresponding to the breath to another state to analyze the data associated with the vibration corresponding to the breath; and in response to the identifying to change the medical device to the other state, automatically changing to the other state utilized to analyze the data associated with the vibration, by supplying power to the connector.

36: The method of any of clauses 32 to 35, wherein receiving the data from the wearable device comprises receiving the data from the wearable device via a connector in response to the wearable device being activated and utilized to detect sound generated via the vibration corresponding to the breath, and wherein the breath that is detected comprises a respiration rate.

37: The method of any of clauses 32 to 36, wherein receiving the data from the wearable device comprises: receiving a signal from a connector comprising a user interface (UI) that receives input; and in response to the identifying that the wearable device is activated and the receiving of the signal, receiving the data from the wearable device and identifying that the data represents the vibration corresponding to the breath.

38: The method of any of clauses 32 to 37, further comprising: prior to receiving the data, pairing the medical device with the wearable device, via a connector, by exchanging, with the wearable device, a code utilized to setup a channel available for communications to be exchanged between the medical device and the wearable device, wherein receiving the data from the wearable device comprises receiving the data from the wearable device via the connector.

39: The method of any of clauses 32 to 38, further comprising: detecting, by an activator of a connector inserted into a port, a tap of the wearable device; receiving a prompt from the wearable device being activated by the tap; transmitting an acknowledgement to the wearable device; and receiving the data in response to receiving the prompt and transmitting the acknowledgement.

40: The method of any of clauses 32 to 39, wherein receiving the data from the wearable device comprises receiving the data from the wearable device via a connector inserted into a port, the receiving of the data ceasing when the connector is disconnected from the port.

### CONCLUSION

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A system, comprising:
a wearable device configured to be worn by a subject, the wearable device comprising:
a sensor configured to detect vibration associated with a breath of the subject; and
a first transceiver configured to transmit a communication signal including data representing the vibration; and
a connector configured to relay signals related to the vibration;
a medical device, comprising:
a port configured to receive the connector and supply power to the connector, the connector comprising a second transceiver configured to receive the data from the first transceiver;
a processor configured to:
generate, via the connector, a communication path with the wearable device being positioned at a distance from the connector that is less than or equal to a threshold distance;
utilize the connector to convert initial signals received from the wearable device to the signals related to the vibration, the signals being relayed by the connector according to a different protocol;
in response to utilizing the connector to convert the initial signals, identify the signals being relayed by the connector and to the medical device according to the different protocol;
receive, via the connector, the data representing the vibration;
in response to the receiving of the data representing the vibration, determine the breath that generates the vibration represented by the data overlaps with an artifact; and
cause output of an alarm.

2. The system of claim 1, wherein the processor is further configured to:
in response to identifying the artifact, identify a classification associated with a historical artifact matching the artifact by a score being above a threshold score;
in response to the identifying of the classification associated with the historical artifact, identify that the classification is associated with the artifact; and
identify an index representing the alarm, the index being associated with the classification;
in response to the identifying of the index, output the alarm; and
cease output of the alarm in response to receiving other data relayed by the connector, the other data representing another vibration associated with the breath of the subject.

3. The system of claim 1, the artifact overlapping with activity associated with a non-invasive blood pressure (NIBP) measurement,
wherein the processor is further configured to:
in response to identifying the artifact, identify a classification of a historical event associated with a historical event matching the artifact by a score being above a threshold score;
in response to the identifying of the classification, identify the historical event as an event associated with the artifact; and
identify an index representing an alarm associated with the event;
in response to the generating of the index, output the alarm; and
cease output of the alarm in response to receiving other data relayed by the connector, the other data representing another vibration associated with the breath of the subject.

4. The system of claim 1, the artifact overlapping with activity represented by an electrocardiogram (ECG) or a non-invasive blood pressure (NIBP) measurement,
wherein generating the communication path comprises:
outputting a notifier to a rescuer of an availability of the connector; and
in response to outputting the notifier and identifying the connector being inserted into the port, generating, via the connector, the communication path;
wherein the processor is further configured to:
cease output of the alarm in response to receiving other data relayed by the connector, the other data representing another vibration associated with the breath of the subject.

5. A medical device, comprising:
a transceiver configured to receive, from a wearable device, data representing vibration identified by the wearable device, the vibration being associated with a breath of a subject; and
a processor configured to:
determine, by analyzing the vibration, that the breath is indicative of a parameter being less than a threshold parameter; and
in response to determining that the data is indicative of the parameter being less than the threshold parameter, initiate an alarm.

6. The medical device of claim 5, wherein the determining that the breath is indicative of the parameter being less than the threshold parameter comprises:
outputting a notifier to a rescuer of an availability of a connector that enables the wearable device to be utilized as a substitute for a stethoscope; and
in response to outputting the notifier and identifying the connector being inserted into a port, generating, via the connector, a communication path between the wearable device and the medical device.

7. The medical device of claim 5, the parameter comprising an electrocardiogram (ECG) parameter,
wherein outputting the alarm comprises:
identifying a historical ECG parameter matching the ECG parameter by a score being above a threshold score;
in response to the identifying of the historical ECG parameter, identifying a classification of a treatment that is associated with the ECG parameter; and
in response to the identifying of the classification, outputting the alarm.

8. The medical device of claim 6, the parameter comprising a non-invasive blood pressure (NIBP) parameter,
wherein outputting the alarm comprises:
identifying a historical NIBP parameter matching the NIBP parameter by a score being less than a threshold score;
in response to the identifying of the historical NIBP parameter, identifying a classification of an event that is associated with the NIBP parameter; and
in response to the identifying of the classification, outputting the alarm.

9. The medical device of claim 5, wherein receiving the data representing the vibration comprises:
detecting, by an activator of a connector inserted into a port, a tap of the wearable device;
receiving a prompt from the wearable device being activated by the tap;
transmitting an acknowledgement to the wearable device; and
receiving the data in response to receiving the prompt and transmitting the acknowledgement.

10. The medical device of claim 5, further comprising:
a sensor configured to generate a signal associated with a connector being inserted into a port, the connector comprising a capability utilized to relay the data received from the wearable device, the wearable device being activated by input received to a user interface (UI),
wherein the processor is further configured to:
detect the connector being inserted into the port in response to receiving the signal from the sensor; and
pair the medical device with the wearable device, via the connector, by causing the transceiver to exchange a code utilized to set up a channel available for communications to be exchanged between the medical device and the wearable device, via the connector.

11. The medical device of claim 5, wherein the transceiver is further configured to receive, via a connector inserted into a port, the data, the receiving of the data ceasing when the connector is disconnected from the port.

12. A method, comprising:
receiving, by a medical device, data from a wearable device in response to identifying that the wearable device is activated to detect vibration corresponding to a breath of a subject on which the wearable device has been placed;
identifying that activity associated with the data overlaps with an artifact; and
in response to the identifying that the activity overlaps with the artifact, initiating, by the medical device, an alarm advising a rescuer to administer, to the subject, a treatment associated with the breath of the subject.

13. The method of claim 12, further comprising:
identifying a pattern of new data that matches, at a level that is greater than a threshold level, another pattern of other data, the other pattern of the other data being associated with activity overlapping with an artifact; and
in response to the identifying of the pattern that matches the other pattern, identifying the pattern is associated with an electrocardiogram (ECG) parameter.

14. The method of claim 12, further comprising:
identifying a pattern of new data; and
identifying the pattern is associated with a non-invasive blood pressure (NIBP) parameter.

15. The method of claim 12, further comprising:
detecting a signature routed from the wearable device and by a connector, in response to identifying the connector being inserted in a port of a medical device and being activated by power supplied by the medical device;
in response to the detecting of the signature, changing the medical device from a first state to a second state, the first state corresponding to an absence of analyzing the vibration corresponding to the breath, the second state corresponding to analyzing the data associated with the vibration corresponding to the breath; and
in response to the changing medical device from the first state to the second state, supplying power to the connector.

16. The method of claim 12, wherein receiving the data from the wearable device comprises receiving the data from the wearable device via a connector in response to the wearable device being activated and utilized to detect sound generated via the vibration corresponding to the breath, and
wherein the breath that is detected comprises a respiration rate.

17. The method of claim 12, wherein receiving the data from the wearable device comprises:
receiving a signal from a connector comprising a user interface (UI) that receives input; and
in response to the identifying that the wearable device is activated and the receiving of the signal, receiving the data from the wearable device and identifying that the data represents the vibration corresponding to the breath.

18. The method of claim 12, further comprising:
prior to receiving the data, pairing the medical device with the wearable device, via a connector, by exchanging, with the wearable device, a code utilized to setup a channel available for communications to be exchanged between the medical device and the wearable device,
wherein receiving the data from the wearable device comprises receiving the data from the wearable device via the connector.

19. The method of claim 12, further comprising:
detecting, by an activator of a connector inserted into a port, a tap of the wearable device;
receiving a prompt from the wearable device being activated by the tap;
transmitting an acknowledgement to the wearable device; and
receiving the data in response to receiving the prompt and transmitting the acknowledgement.

20. The method of claim 12, wherein receiving the data from the wearable device comprises receiving the data from the wearable device via a connector inserted into a port, the receiving of the data ceasing when the connector is disconnected from the port.
